(19) United States
(12) Patent Application Publication
(10) Pub. No.: US 2011/0275591 A1
(43) Pub. Date: Nov. 10, 2011

(54) COCKTAIL FOR MODULATION OF ALZHEIMER'S DISEASE

(75) Inventor: Curt Hendrix, West Lake Village, CA (US)

(73) Assignee: Concourse Health Sciences LLC, West Lake Village, CA (US)

(21) Appl. No.: 13/104,588

(22) Filed: May 10, 2011

Related U.S. Application Data
(63) Continuation of application No. PCT/US2009/046149, filed on Jun. 3, 2009.
Application No. PCT/US2009/046149 is a continuation-in-part of application No. 12/325,842, filed on Dec. 1, 2008, now abandoned.
Application No. 12/325,842 is a continuation-in-part of application No. 12/149,075, filed on Apr. 25, 2008, now abandoned.
Application No. 12/149,075 is a continuation of application No. 11/293,425, filed on Dec. 1, 2005, now abandoned.
Application No. 11/293,425 is a continuation-in-part of application No. 11/002,750, filed on Dec. 1, 2004, now abandoned.
Application No. 11/002,750 is a continuation-in-part of application No. 11/116,997, filed on Apr. 27, 2005, now abandoned.
(60) Provisional application No. 60/996,702, filed on Nov. 30, 2007.
(60) Provisional application No. 60/632,681, filed on Dec. 1, 2004.

(51) Int. Cl. A61K 31/4525; A61P 25/28; A61K 31/714; A61K 31/519; A61K 31/675; A61K 31/51

(52) U.S. Cl. 514/52; 514/321; 514/89; 514/276; 514/249

(57) ABSTRACT

Formulations for the prevention and treatment of neurological diseases and cognitive deficiencies, i.e., Alzheimer's Disease (AD), Parkinson's Disease, amyotrophic lateral sclerosis, mild cognitive impairment and other types of dementia, comprise therapeutically effective amounts of curcumin, piperine, epigallocatechin-3-gallate (EGCG) and one or more of N-acetylcysteine, benfotiamine and alpha-lipoic acid. The combination addresses some or all of the pathways which can result in neurological deficiencies, degeneration and diseases.

This application is a Continuation of International Application No. PCT/US2009/046149, filed Jun. 3, 2009, which claims priority to U.S. application Ser. No. 12/325,842, filed Dec. 1, 2008. This application is also a Continuation-in-Part of co-pending U.S. application Ser. No. 12/325,842, filed Dec. 1, 2008, which claims the benefit of U.S. Provisional Application No. 60/996,702, filed Nov. 30, 2007; and which also is a Continuation-in-Part of U.S. application Ser. No. 12/149,075, filed Apr. 25, 2008, which is a Continuation of U.S. application Ser. No. 11/293,425, filed Dec. 1, 2005, which is a Continuation-in-Part of U.S. application Ser. No. 11/002,750, filed Dec. 1, 2004 and of U.S. application Ser. No. 11/116,997, filed Apr. 27, 2005, and which claims benefit of U.S. Provisional Application No. 60/632,681, filed Dec. 1, 2004. Each of these applications is hereby incorporated by reference in its entirety and for all purposes.

### FIELD OF THE INVENTION

This application is directed to new formulations for reducing phosphorylation of tau proteins or the presence of amyloid beta plaques to prevent and treat cognitive and neurological disorders such as Alzheimer's Disease (AD). Other neurological disorders treatable according to the invention include amyotrophic lateral sclerosis; mild cognitive impairment; frontotemporal dementia, for example associated with Parkinsonism linked to chromosome 17; frontotemporal lobar degeneration, also referred to as Pick's disease; progressive supranuclear palsy; encephalomyelitis; multiple sclerosis; or corticobasal degeneration; Parkinson's disease; and other types of dementia. The formulations comprise, e.g., a therapeutically effective amount of a combination of curcumin, piperine, epigallocatechin-3-gallate (EGCG) and one or more of N-acetylcysteine, benfotiamine and alpha-lipoic acid. The combination addresses some or all of the pathways which can result in neurological deficiencies, degeneration and diseases.

### BACKGROUND

Alzheimer's disease (AD) is the leading cause of dementia in the elderly. It is generally characterized by a loss of cognitive abilities, including memory, and a rapid deterioration in personality and the ability to care for oneself. Over 5 million Americans are currently diagnosed with AD, and this number could triple over the coming decades as the population ages. One in 10 people aged 65 and over, and around 1 in 2 over the age of 85, develop the disease. Researchers have generally found that the disease itself manifests with the appearance of several hallmark pathologies, including the accumulation of amyloid β (Aβ) plaques and hyperphosphorylated tau proteins. Large inflammatory responses are also seen, along with evidence of oxidative damage. Extensive synaptic and neuronal loss is also frequently observed in AD patients.

The standard of care for patients with AD is treatment with anticholinesterase inhibitors. Cholinesterase inhibitors increase the synaptic availability of the neurotransmitter acetylcholine by preventing it from breaking down. Anticholinesterase inhibitors act to slow progression of the disease, particularly deterioration in cognitive function and overall functioning, and often delay the need for institutionalization by several months. Unfortunately, the effect of anticholinesterase inhibitors is only temporary. Memantine, an N-methyl-D-aspartate receptor antagonist, has also been used in an attempt to treat AD. However, anticholinesterase inhibitors and memantine, to the extent they have any impact on AD, bring about only a temporary effect on the symptoms of AD, and have not been shown to be disease-modifying. It is believed that no treatment currently in use has been shown to prevent, halt, or reverse the neurodegenerative process.

A successful treatment for AD will have to address both the accumulation of aggregating biomolecules, such as Aβ and hyperphosphorylated tau, as well as the loss of synapses and neurons. One promising approach is to prevent the development of pathologies in the first place, which is likely to at least delay the onset of the disease. Such a treatment should be safe for prolonged use, and well tolerated by the general population.

### SUMMARY

There is a great need for a significant breakthrough in Alzheimer's prevention and treatment. According to the present invention, a “cocktail” of medicines or ingredients can successfully delay onset or progression of Alzheimer's disease. In particular, a cocktail composed of an inventive combination of standardized herbal extracts, vitamins, and minerals has been found to impact the biochemical and pathophysiological processes involved in Alzheimer's disease.

In some embodiments, the invention provides a standardized cocktail, which includes, for example, extracts of tumeric, green tea, black pepper, vitamins and other nutritive ingredients. The cocktail has been shown to reduce the prevalence of pathophysiological markers of AD, such as tau protein hyperphosphorylation and/or Aβ plaque precursor moieties, and also treating the cognitive and behavioral effects of AD, as demonstrated in, for example, a novel transgenic mouse model of Alzheimer's disease.

In one aspect, the invention provides uses of a cocktail comprising curcumin, piperine, epigallocatechin-3-gallate and one or more of N-acetylcysteine, benfotiamine and alpha-lipoic acid for treating a cognitive or neurological disorder. The cocktail treats the cognitive or neurological disorder by, for example, reducing the prevalence of tau protein hyperphosphorylation, or by reducing the prevalence of an Aβ plaque precursor moiety. In some embodiments, the cognitive or neurological disorder is Alzheimer's disease. The cognitive or neurological disorder can also be, for example, amyotrophic lateral sclerosis, mild cognitive impairment, Parkinson's disease, frontotemporal dementia with Parkinsonism linked to chromosome 17, Pick's disease, progressive supranuclear palsy, multiple sclerosis, encephalomyelitis, and corticobasal degeneration. Reducing the prevalence of tau protein hyperphosphorylation and/or an Aβ plaque precursor moiety can lead to improvement in one or more cognitive symptoms including, without limitation, memory loss, personality change, agitation, disorientation, loss of coordination, inability to care for one's self, and combinations thereof. In some embodiments, reducing the prevalence of an Aβ plaque precursor moiety leads to a reduction in the prevalence of Aβ plaques. The Aβ plaque precursor moiety can be, for example, Aβ 42, C99, the low molecular weight oligomeric Aβ species Aβ*56, and combinations thereof. In some embodiments, the tau protein hyperphosphorylation comprises phosphorylation of tau protein at threonine 231. The cocktail can be administered in unit dosage form comprising one, or more than one, unit dosages daily.

In another aspect, the invention provides cocktails. In some embodiments, the cocktails comprise curcumin, piperine, epigallocatechin-3-gallate and one or more of N-acetylcysteine, benfotiamine and alpha-lipoic acid for use in treating a cognitive or neurological disorder. The cocktail can treat the cognitive or neurological disorder by reducing the prevalence of tau protein hyperphosphorylation, or by reducing the prevalence of an Aβ plaque precursor moiety. The cognitive or neurological disorder can be, for example, Alzheimer's disease, or amyotrophic lateral sclerosis, mild cognitive impairment, Parkinson's disease, frontotemporal dementia with Parkinsonism linked to chromosome 17, Pick's disease, progressive supranuclear palsy, multiple sclerosis, encephalomyelitis, and corticobasal degeneration. Reducing the prevalence of tau protein hyperphosphorylation or an Aβ plaque precursor moiety leads to improvement in one or more cognitive symptoms including, for example, memory loss, personality change, agitation, disorientation, loss of coordination, inability to care for one's self, and combinations thereof. Reducing the prevalence of an Aβ plaque precursor moiety can lead, for example, to a reduction in the prevalence of Aβ plaques. The Aβ plaque precursor moiety can be, for example, Aβ 42, C99, the low molecular weight oligomeric Aβ species Aβ*56, and combinations thereof. In some embodiments, the tau protein hyperphosphorylation comprises phosphorylation of tau protein at threonine 231. The cocktail can be administered in unit dosage form comprising one, or more than one, unit dosages daily.

In still another aspect, the invention provides uses of a cocktail comprising, for example, curcumin, piperine, epigallocatechin-3-gallate and one or more of N-acetylcysteine, benfotiamine and alpha-lipoic acid for reducing the prevalence of tau protein hyperphosphorylation. In some embodiments, the tau protein hyperphosphorylation comprises, for example, phosphorylation of tau protein at threonine 231. In yet another aspect, the invention provides uses of a cocktail comprising curcumin, piperine, epigallocatechin-3-gallate and one or more of N-acetylcysteine, benfotiamine and alpha-lipoic acid for reducing the prevalence of an Aβ plaque precursor moiety.

### BRIEF DESCRIPTION OF THE DRAWINGS

FIGS. 1A-1F present a series of graphs depicting the effects of a cocktail according to an embodiment of the present invention on performance in the Morris water maze tests in the Tg2576 transgenic mouse model.

FIG. 2 depicts the effects of a cocktail according to an embodiment of the present invention on novel object recognition in the Tg2576 transgenic mouse model.

FIGS. 3A-3F present a series of graphs depicting the effects of a cocktail according to an embodiment of the present invention on several biochemical markers related to the etiology of AD in the Tg2576 transgenic mouse model.

FIG. 4A-4D present a series of graphs depicting the effects of a cocktail according to an embodiment of the present invention on performance in the Morris water maze (A-B) and on levels of several biochemical markers related to the etiology of AD (C-D) in the 3xTg-AD mouse model.

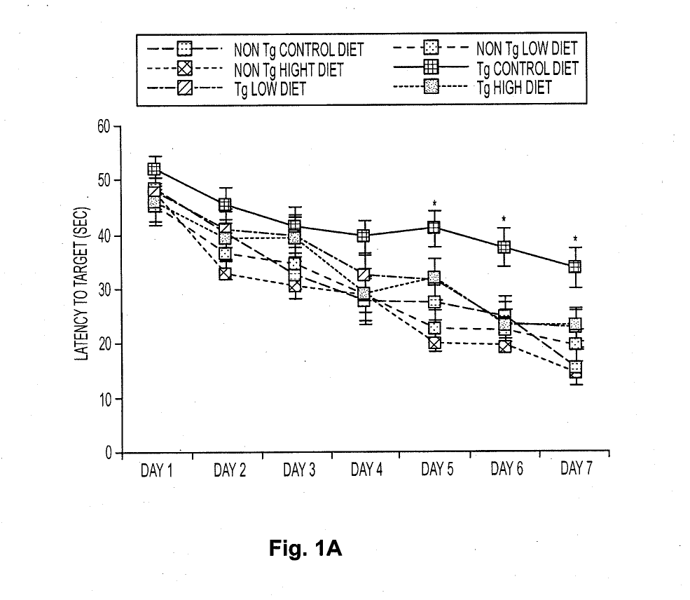

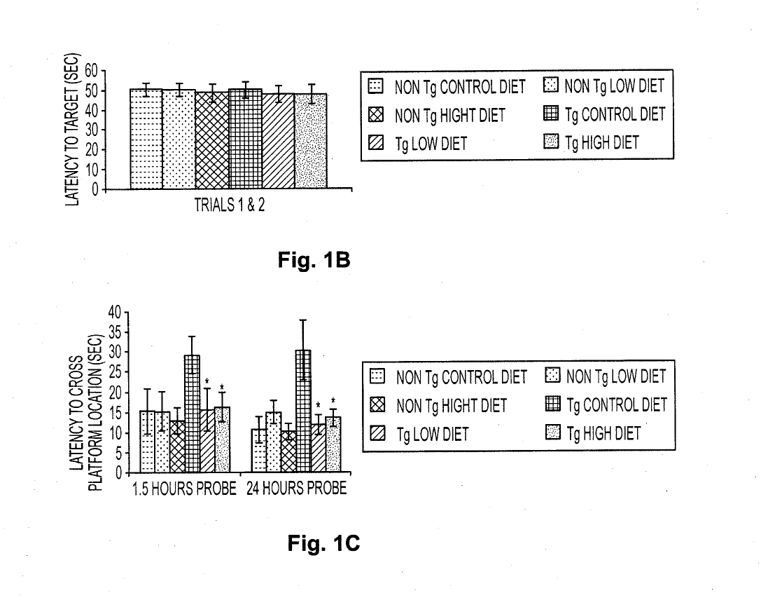

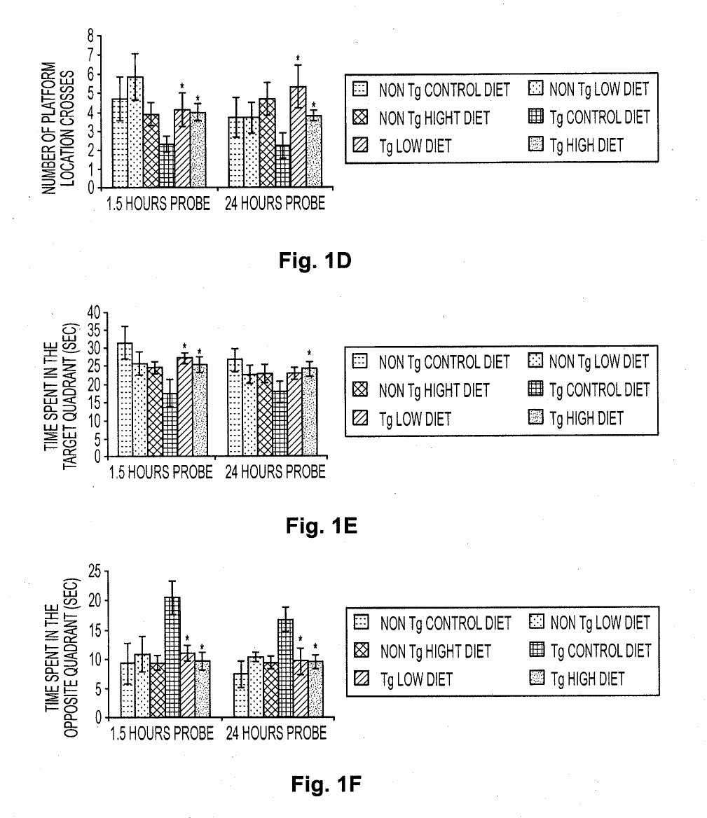

### DETAILED DESCRIPTION

Embodiments of the invention are discussed in detail below. In describing embodiments, specific terminology is employed for the sake of clarity. However, the invention is not intended to be limited to the specific terminology so selected. A person skilled in the relevant art will recognize that other equivalent parts can be employed and other methods developed without parting from the spirit and scope of the invention. All references cited herein are incorporated by reference as if each had been individually incorporated. Percentages are generally by weight, unless stated or suggested otherwise.

In some embodiments, the invention provides a medical food cocktail that can slow, halt or reverse the development of Alzheimer's disease or another neurological or cognitive disorder during the early stages of the disease. The cocktail is composed of nutritional ingredients that are demonstrated to beneficially impact the biochemical or physiological processes involved in Alzheimer's disease, for example reducing the prevalence of Aβ plaques and tau hyperphosphorylation, as demonstrated in a well-regarded mouse model of AD. It is believed to be the first time such effects have been demonstrated, and/or demonstrated in this magnitude and scope. This beneficial impact on biochemical and/or physiological processes results in improvement or lack of decline in cognitive functions. These ingredients are all currently listed as Generally Recognized As Safe (GRAS) by the FDA, or are self-affirmed as GRAS ingredients, or in common use as dietary supplements. The inventive compositions have been found to be beneficial in preventing, reducing the severity of, or reversing various neurological diseases or cognitive disorders, including but not limited to Alzheimer's disease, Parkinson's disease and mild cognitive impairment.

As used herein, “treat” refers to preventing, curing, reversing, attenuating, alleviating, minimizing, suppressing or halting at least one of the symptoms or deleterious effects of the diseases, disorders or conditions described herein. “Treatment” encompasses both therapeutic treatment and prophylactic or preventative measures and does not demand a cure. Those in need of treatment include those already with the disorder as well as those in which the disorder is to be prevented. Hence, the patient to be treated may have been diagnosed as having the disorder or may be predisposed or susceptible to the disorder. “Treating” also encompasses reducing the symptoms of cognitive decline, such as memory loss, personality change, agitation, disorientation, loss of coordination, inability to care for one's self, and combinations thereof.

“Effective” or “therapeutically effective” means sufficient to cause at least one of a patient's symptoms to decrease in frequency and/or intensity; to slow the rate of increase in frequency and/or intensity of one or more symptoms; or to maintain a relatively static level of frequency and/or intensity when an increase would otherwise be expected. The symptoms that are thus affected can include, for example, one or more adverse cognitive or physiological symptoms.

“Cognitive or neurological disorder” encompasses diseases and disorders that may be characterized by a decline in cognitive function, for example disorders that cause memory loss, and disorders characterized by a loss of neurological function, including but not limited to disorders that affect the function of the neurological system overall or that result in neuronal death or in a decline of neuronal health. For example, “cognitive or neurological disorder” includes Alzheimer's disease; amyotrophic lateral sclerosis (ALS); mild cognitive impairment; frontotemporal dementia, for example associated with Parkinsonism linked to chromosome 17; frontotemporal lobar degeneration, also referred to as Pick's disease; progressive supranuclear palsy (PSP); encephalomyelitis; multiple sclerosis (MS); Parkinson's disease; or corticobasal degeneration (CBD); or any symptom or symptoms associated with these or other disorders.

“Administer” means to deliver one or more doses of one of the compositions to a patient. The methods of the present invention can involve administration of the composition by any means and via any route of administration that is consistent with effective treatment of one or more of the diseases described herein. For example, the methods can involve administering the compositions orally.

As used herein, “patient” encompasses a mammal, such as a human, that is diagnosed with one of the diseases, disorders or conditions described herein, or is predisposed to at least one of the diseases, disorders or conditions described herein. The compositions of the invention can be administered to any mammal that can experience the beneficial effects of the compositions and methods of the invention. Any such mammal is considered a “patient.” Such patients include humans and non-humans, such as humans, domestic and farm animals, and zoo, sports, or pet animals, such as dogs, horses, cats, cows, mice, rats, etc.

“Adverse cognitive symptom” encompasses any undesirable cognitive symptom that can be effectively treated by the compositions and methods of the present invention. Examples of adverse cognitive symptoms include, without limitation, memory loss, personality change, agitation, disorientation, loss of coordination, and inability to care for one's self.

“Adverse physiological symptom” encompasses any undesirable physiological symptom that can be effectively treated by the compositions and methods of the present invention. Examples of adverse physiological symptoms include, without limitation, formation or accumulation of amyloid (i.e., Aβ) plaques, formation or accumulation of tau protein tangles, tau protein hyperphosphorylation, tau protein phosphorylation at threonine 231, microtubule destabilization, and/or synaptic loss.

As used herein, “cocktail” encompasses a composition comprising two or more of the ingredients disclosed herein. As used herein, “cocktail,” “combination” and “combination diet” can be used interchangeably.

“Hyperphosphorylation” encompasses any increase in phosphorylation over normal levels, i.e., above those that would occur in a healthy patient, for example those levels associated with a cognitive or neurological disorder such as AD. As would be understood by one of ordinary skill in the art, “normal” levels of phosphorylation may vary significantly from patient to patient, and what constitutes “normal” phosphorylation or “hyperphosphorylation” will vary with the context. As used herein, “hyperphosphorylation” also encompasses abnormal phosphorylation, whether as to level of phosphorylation, site or sites of phosphorylation, or otherwise differing from that which occurs under normal circumstances, including types of abnormal phosphorylation associated with a cognitive or neurological disorder such as AD. For example, “hyperphosphorylation” encompasses abnormal phosphorylation at threonine 231, a particularly toxic form of tau hyperphosphorylation that can lead to neuronal dysfunction and neuronal death. The threonine 231 residue shows abnormal phosphorylation in patients with Alzheimer's disease, although it is also phosphorylated, to a certain extent, in normal brain.

Cocktails

In some embodiments, the invention provides cocktails for use in treating a cognitive or neurological disorder. The cocktails disclosed herein can comprise, for example, curcumin, piperine, epigallocatechin-3-gallate and one or more of N-acetylcysteine, benfotiamine and alpha-lipoic acid.

The cognitive or neurological disorder can be, for example, Alzheimer's disease, though any disease, disorder or condition that is characterized by Aβ plaques and/or tau tangles can be treated with the cocktails described herein. Examples of such cognitive or neurological disorders include, without limitation, amyotrophic lateral sclerosis (ALS); mild cognitive impairment; frontotemporal dementia, for example associated with Parkinsonism linked to chromosome 17; frontotemporal lobar degeneration, also referred to as Pick's disease; PSP; encephalomyelitis; MS; Parkinson's disease; or CBD.

The compositions and methods disclosed herein can be used to treat, e.g., Alzheimer's disease. While a single cause for Alzheimer's disease has not been identified, the brain of people diagnosed with AD typically exhibit sticky plaques composed of beta amyloid protein deposits (i.e., plaques) as well as tau protein tangles resulting from hyperphosphorylation of tau proteins. The compositions and methods of the present invention bring about the prevention of plaques and tangles, and/or their reversal/reduction if formed. Compositions according to the invention have been shown to reduce amyloid plaques, tau protein tangles, microtubule destabilization, synaptic loss, and tau protein hyperphosphorylation, and can prevent cognitive decline to effectively treat memory loss, personality change, agitation, disorientation, inability to care for one's self, and loss of coordination in AD patients.

Beta amyloid (also referred to herein as Aβ or Abeta) peptides have been the central focal point of AD research for over a decade. The presence of Aβ peptides and plaque formation therefrom is generally considered to be an upstream causative factor. Evidence for this position derives from molecular genetic studies of the three genes—amyloid precursor protein (APP), presenilin 1 (PS1) and presenilin 2 (PS2)—that underlie some AD cases, as they all modulate some aspect of Aβ metabolism, increasing the propensity for Aβ to aggregate. In addition, the E4 variant of the apolipoprotein E (Apo E4) gene, which is a modifier gene linked to late-onset disease, affects the rate of Aβ aggregation. Apo E4 may facilitate β-sheet formation from Aβ peptides, thus facilitating the formation of plaques from otherwise-soluble peptides. Apo E4 occurs in about 40 percent of all people who develop late-onset AD and is present in about 25 to 30 percent of the population. People with AD are more likely to have an Apo E4 allele than people who do not develop AD. However, many people with AD do not have an Apo E4 allele.

APP is a neuronal transmembrane protein that is critical to neuronal growth, survival and post-injury repair. APP can be broken down by the enzyme α-secretase into the peptide moiety C83 and an N-terminal fragment, or by the enzyme β-secretase into the moiety C99 and an N-terminal fragment. When C99 is produced, it can be further cleaved by gamma-secretase to produce Aβ 42, which is associated with the formation of amyloid plaques. The α-secretase pathway is not currently shown to lead to the formation of any compounds involved in the etiology of AD.

Amyloid plaques form extracellularly. Formation of amyloid plaques is associated with, e.g., disruption of calcium-ion homeostasis, induction of neuronal apoptosis, inhibition of the function of certain enzymes, and interference with glucose utilization by neurons. Amyloid plaques may also be associated with disease states by stimulating the formation of reactive oxygen species.

AD is also characterized by the formation of tau tangles. The tau protein is encoded by a single gene (MAPT) located on chromosome 17, although it is alternatively spliced to yield 6 major protein isoforms in the adult human brain. The tau gene contains 15 exons, and exons 2, 3, and 10 can be alternatively spliced. Four imperfect tandem repeats are encoded by exons 9-12, hence, alternative splicing of exon 10 yields isoforms with 3 or 4 repeat domains (3R and 4R tau), depending if exon 10 is absent or present, respectively. Alternative splicing of exons 2 and 3 yields variants containing zero (0N), one (1N), or two (2N) inserts at the amino terminus, such that 6 tau isoforms are formed: 3R0N, 3R1N, 3R2N, 4R0N, 4R1N, and 4R2N. In the adult human brain, the proportion of 3R to 4R tau is ˜1:1, whereas in the adult mouse brain, 4R tau is the only tau isoform present. Tauopathies can be further classified based on whether tangles are comprised of 3R or 4R tau isoforms. For example, in AD, both 3R and 4R tau accumulate in neurofibrillary tangles; other disorders are marked by only 3R tau (e.g., Pick's disease) or 4R tau (e.g., PSP and CBD). In AD, tau pathology is restricted to neurons, but in certain other tauopathies, such as 4R tauopathies CBD and PSP, tau inclusions are also observed in glia.

In its normal state, tau is a soluble protein whose function is to facilitate the proper functioning of the cytoskeleton by promoting microtubule assembly and stabilization. Pathological tau protein, by contrast, exhibits altered solubility properties, forms filamentous structures, and is hyperphosphorylated or abnormally phosphorylated at certain residues. Pathological tau shows reduced affinity for microtubules, intering with its ability to promote microtubule assembly. Hyperphosphorylation of tau is believed to be an early event that precedes assembly into PHFs.

Hyperphosphorylated or abnormally phosphorylated tau brings about the formation of tau tangles. Tau tangles are characterized by the formation of paired helical filaments (PHF), which aggregate into neurofibrillary tangles (NFT). NFTs are filamentous inclusions that accumulate in selective neurons in the brains of individuals with AD, but they also occur in other neurodegenerative disorders including frontotemporal dementia with Parkinsonism linked to chromosome 17 (FTDP-17), Pick's disease, PSP and CBD. Formation of tangles is thought to interfere with the proper functioning of the neuronal cytoskeleton, in some cases leading to the collapse of the microtubule network, affecting intracellular transport and other cellular functions. NFT formation is also associated with synaptic loss, neuritic atrophy, and neuronal death. More than 30 phosphorylation sites are known to exist in PHF tau. In contrast, in healthy brains, tau is generally phosphorylated at between eight and ten of these residues. A growing number of investigators now view tau hyperphosphorylation as a central cause of AD pathology.

Formation of Aβ is thought to be capable of contributing to the initiation of tau hyperphosphorylation in some cases. For example, extracellular amyloid can lead to dysregulation of cyclin-dependent kinase Cdk5, an enzyme involved in brain development, and this dysregulation may lead to tau hyperphosphorylation.

Other evidence suggests that tau and amyloid pathologies arise independently from a common underlying mechanism. For example, some studies suggest that phosphorylated APP is more likely than non-phosphorylated forms to be processed via pathways that lead to Aβ formation. Kinases that can phosphorylate APP can also phosphorylate tau. It has also been suggested that another common factor, such as loss of wnt signaling, might induce both plaques and tangles. Wnt signaling is a form of cell-to-cell signaling that involves a complex network of proteins, and is involved in normal physiological processes in adult animals, though they are also thought to be involved in embryogenesis and cancer.

It is also clear that tau hyperphosphorylation, and associated neuronal pathologies, can occur independently of Aβ plaque formation. For example, in frontotemporal dementia, which is characterized by neurodegeneration and dementia, tau tangles form in the absence of Aβ plaques. In some transgenic mouse models, amyloid generation does not induce the predicted cascade that is thought to lead to tau hyperphosphorylation, suggesting that tau hyperphosphorylation arises through other mechanisms in these models. In addition, those portions of the human brain that are most susceptible to neurofibrillary changes, such as those brought about by tau hyperphosphorylation, are generally most resistant to β-amyloid deposition. For example, the entorhinal cortex and the hippocampus are affected by neurofibrillary pathology early on in the progression of AD, but they generally do not develop Aβ plaques until the late stages of the disease. Oxidative stress has been shown to lead to the disruption of biochemical pathways which, in turn, leads to neuronal degradation, possibly via tau hyperphosphorylation brought about independently of Aβ plaque formation.

Using the cocktails and methods disclosed herein, the cognitive or neurological disorder is treated because the cocktails have been shown to be effective at reducing the prevalence of one or more physiological markers of cognitive or neurological disorders such as AD. For example, in some embodiments of the invention, a cocktail comprising curcumin, piperine, epigallocatechin-3-gallate and one or more of N-acetylcysteine, benfotiamine and alpha-lipoic acid can be used in treating a cognitive or neurological disorder because, among other things, it reduces the prevalence of tau protein hyperphosphorylation, for example by reducing the prevalence of phosphorylation of tau protein at threonine 231, an important physiological marker of AD as well as other cognitive or neurological diseases.

As used herein, “reduce the prevalence” encompasses, for example, reducing the number, concentration, level, or other measurement of the physiological marker as compared with the same value as measured before the start of treatment with the cocktail; slowing the rate of increase of the marker; maintaining a steady level when an increase would have been expected in the absence of treatment; and/or preventing, inhibiting or interfering with the onset or development of the physiological marker. As used herein, “prevalence” encompasses presence as well as formation.

In some embodiments, the cocktail treats a cognitive or neurological disorder by reducing the prevalence of tau protein hyperphosphorylation. Examples of tau hyperphosphorylation include, for example, abnormal phorphorylation at threonine 231, and/or an increase in tau phosphorylation levels, and/or an increase in the number of phosphorylation sites, as compared to those seen under normal conditions.

According to some embodiments, reducing the prevalence of hyperphosphorylation of tau protein, for example at threonine 231, can treat a neurological or cognitive disorder and lead to improvement in one or more cognitive symptoms. Such symptoms can include, for example, memory loss, personality change, agitation, disorientation, loss of coordination, inability to care for one's self, and combinations thereof.

In some embodiments, the cocktails disclosed herein treats a cognitive or neurological disorder by reducing the prevalence of an Aβ plaque precursor moiety. As used herein, “Aβ plaque precursor moiety” means any moiety that can or does contribute to the formation of Aβ plaques. Examples of Aβ plaque precursor moieties include, for example, the peptides Aβ 42 and C99, as well as other low molecular weight oligomeric species such as, for example, Aβ*56.

Reducing the prevalence of an Aβ plaque precursor moiety can lead to a reduction in the prevalence of Aβ plaques, and thus to a reduction in adverse physiological events associated with the presence or formation of Aβ plaques. And as with tau hyperphosphorylation, reducing the prevalence of an Aβ plaque precursor moiety can lead to improvement in one or more cognitive symptoms associated with cognitive or neurological disorders. Such symptoms can include, for example, memory loss, personality change, agitation, disorientation, loss of coordination, inability to care for one's self, and combinations thereof.

The cocktails can also include one or more additional ingredients such as, for example, vitamin B₅, vitamin B₆, vitamin B₁₂, folic acid, vitamin C, vitamin E, L-carnosine, and proteolytic enzymes.

In some embodiments, the compositions of the present invention are to be administered at a dosage of from about 15 mg/kg/day to about 500 mg/kg/day. In some embodiments, the compositions of the present invention can be administered in a dosage of about 1050 mg/day to about 35,000 mg/day. The dosage to be administered can comprise, for example, curcumin in an amount of at least about 5 mg/kg patient body weight; or from about 15 to about 170 mg/kg patient body weight; or from about 17 to about 50 mg/kg body weight; or up to or at least about 15, 16, 17, 18, 19, 20, 21, 22, 23, 24, 25, 30, 35, 40, 50, 50, 55 mg/kg patient body weight or more. For example, the dosage of curcumin can be at least about 350 mg; from about 1050 to about 12000 mg; from about 1200 to about 3600 mg; or up to or at least about 1000, 1050, 1100, 1150, 1200, 1250, 1300, 1350, 1400, 1450, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000 mg curcumin or more. The dosage to be administered can comprise, for example, EGCG in an amount of at least about 3.0 mg/kg patient body weight; or from about 7.5 to about 85 mg/kg patient body weight; or from about 8.5 to about 25 mg/kg body weight; or up to or at least about 7.0, 7.5, 8.0, 8.5, 9.0, 9.5, 10, 11, 12, 13, 14, 15, 16, 17, 18, 19, 20, 21, 22, 23, 24, 25, 26, 27, 28, 29, 30 mg/kg body weight or more. For example, the dosage of EGCG can be at least about 210 mg; from about 525 to about 6000 mg; from about 600 to about 1800; or up to or at least about 500, 550, 600, 650, 700, 750, 800, 850, 900, 950, 1000, 1100, 1200, 1300, 1400, 1500, 1600, 1700, 1800, 1900, 2000 mg EGCG or more. The dosage to be administered can comprise, for example, N-acetylcysteine in an amount of at least about 2.5 mg/kg patient body weight, or from about 6.4 to about 71 mg/kg body weight, or from about 7.0 to about 21 mg/kg body weight; or up to or at least about 6.0, 6.5, 7.0, 7.5, 8.0, 8.5, 9.0, 9.5, 10, 11, 12, 13, 14, 15, 16, 17, 18, 19, 20, 21, 22, 23, 24, 25 or more mg/kg body weight. For example, the dosage of N-acetylcysteine can be at least about 175 mg; or from about 450 to about 5000 mg, or from about 500 to about 1500, or up to or at least about 400, 450, 500, 550, 600, 650, 700, 750, 800, 850, 900, 950, 1000, 1100, 1200, 1300, 1400, 1500, 1600, 1700, 1800, 1900, 2000 or more mg N-acetylsysteine. The dosage to be administered can comprise, for example, α-lipoic acid in an amount from about of at least about 1.5 mg/kg patient body weight; or from about 3.5 mg/kg patient body weight to about 43 mg/kg body weight; or from about 4 mg/kg body weight to about 13 mg/kg body weight; or up to or at least about 3.0, 3.5, 4.0, 4.5, 5.0, 5.5, 6.0, 6.5, 7.0, 7.5, 8.0, 8.5, 9.0, 9.5, 10, 11, 12, 13, 14, 15 mg/kg patient body weight or more. For example, the dosage of α-lipoic acid can be at least about 100 mg; from about 270 mg to about 3000 mg; from about 300 to about 900 mg; or up to or at least about 200, 250, 300, 350, 400, 450, 500, 550, 600, 700, 800, 900, 1000, 1100, 1200 or more mg α-lipoic acid. For example, the R form of α-lipoic acid can be used. The dosage to be administered can comprise, for example, piperine in an amount of at least about 0.05 mg/kg patient body weight; from about 0.1 up to about 1.5 mg/kg patient body weight; or from about 0.14 to about 0.43 mg/kg body weight; or up to or at least about 0.1, 0.11, 0.12, 0.13, 0.14, 0.15, 0.16, 0.17, 0.18, 0.19, 0.2, 0.25, 0.3, 0.35, 0.4, 0.45, 0.5 mg/kg body weight or more. For example, the dosage of piperine can be at least about 3.5 mg; or from about 9 mg to about 100 mg; or about 10 mg to about 30 mg; or up to or at least about 1, 2, 3, 4, 5, 6, 7, 8, 9, 10, 11, 12, 13, 14, 15, 16, 17, 18, 19, 20, 25, 30, 35, 40 mg or more piperine. Piperine can be provided in the form of, for example, Bioperine®, which comprises about 98% piperine. The dosage to be administered can comprise, for example, vitamin B₁ in an amount of at least about 0.5 mg/kg patient body weight; or from about 1.2 mg/kg patient body weight to about 14 mg/kg body weight; or about 1.4 mg/kg body weight to about 4.3 mg/kg body weight; or up to or at least about 1.0, 1.1, 1.2, 1.3, 1.4, 1.5, 1.6, 1.7, 1.8, 1.9, 2.0, 2.3. 2.5, 2.8, 3.0, 3.5, 4.0, 4.5, 5.0 mg/kg body weight or more. For example, the dosage of vitamin B₁ can be at least about 35 mg; or from about 90 to about 1000 mg; or from about 100 to about 300 mg; or up to or at least about 75, 80, 90, 100, 110, 120, 130, 140, 150, 160, 170, 180, 190, 200, 220, 240, 260, 280, 300, 325, 350, 375, 400 mg or more vitamin B₁. Vitamin B₁ can be provided in the form of, for example, benfotiamine. These dosages can be administered, for example, on a daily basis.

The dosage to be administered can comprise, for example, vitamin B₆ of at least about 0.6 mg/kg patient body weight, or from about 1.2 mg/kg patient body weight to about 14.3 mg/kg body weight, or from about 1.4 mg/kg body weight to about 43 mg/kg body weight; or at least about 40 mg, or from about 90 mg to about 1000 mg, or from about 100 mg to about 300 mg. Any form of vitamin B₆ can be used. The dosage to be administered can comprise, for example, vitamin E in an amount of at least about 2.0 mg/kg patient body weight, or from about 4.5 to about 50 mg/kg patient body weight, or from about 5 mg/kg body weight to about 15 mg/kg body weight; or at least about 150 mg, or from about 315 to about 3500 mg, or from about 350 to about 1050 mg. Vitamin E can be administered in the form of, for example, tocopheryl succinate. The dosage to be administered can comprise, for example, vitamin B₁₂ in an amount of at least about 1.0 micrograms/kg patient body weight, or from about 2.5 to about 28.5 micrograms/kg patient body weight, or from about 2.8 to about 8.5 micrograms/kg body weight; or at least about 70 micrograms, or from about 180 to about 2000 micrograms, or from about 200 to about 600 micrograms. Any form of vitamin B₁₂ can be used. Vitamin B₁₂ can be provided using a source that includes about 1% vitamin B₁₂, in which case the source will be included in an amount about 100 times the amounts set forth above for vitamin B₁₂. The dosage to be administered can comprise, for example, folic acid in an amount of at least about 3.0 micrograms/kg patient body weight, or from about 10 to about 115 micrograms/kg patient body weight, or from about 11 to about 35 micrograms/kg patient body weight; or at least about 200 micrograms, or from about 720 micrograms to about 8000 micrograms, or from about 800 micrograms to about 2400 micrograms. Folic acid can be provided in a source that includes about 10% folic acid, in which case the source will be included in an amount about 10 times the amounts set forth above for folic acid. The dosage to be administered can comprise, for example, vitamin C in an amount of at least about 0.3 mg/kg patient body weight, or from about 0.6 to about 7.2 mg/kg patient body weight, or from about 0.7 to about 2.1 mg/kg body weight; or at least about 20 mg, or from about 45 to about 500 mg, or from about 50 to about 150 mg. These dosages can be administered, for example, on a daily basis.

The composition can be prepared in a form that provides a daily oral dose comprising, for example, at least about 1200 mg curcumin, at least about 10 mg piperine, at least about 600 mg epigallocatechin-3-gallage and one or more of at least about 500 mg N-acetylcysteine, at least about 100 mg benfotiamine, and at least about 300 mg α-lipoic acid. The compositions can further comprise one or more of 100 mg vitamin B₆, 200 micrograms vitamin B₁₂, 800 micrograms folic acid, 50 mg vitamin C and/or 350 mg vitamin E. Compositions according to the present invention are therapeutically effective to treat a cognitive or neurological disorder, such as, for example, AD, in a patient.

As will be appreciated, a composition comprising the daily amounts recited herein can be present in a single dosage unit, e.g. a single tablet or capsule, or two, three or more dosage units. In the case of a single dosage unit, a minimum daily dose could be taken once daily. In the case of multiple dosage units, the totally daily dosage could be administered once daily, by taking multiple dosage units at the same time, or individual dosage units could be taken at different times during the day to provide the indicated dosage on a daily basis.

An example composition, in which concentrations are expressed in percent gross weight, is listed in Table 1.

TABLE 1
Example Cocktail

| | | Exemplary Active | Concentration |
|---|---|---|---|
| | Concentration | Ingredient* (Conc. | Active (% |
| Ingredient | (%) | in Ingredient**) | Total Actives) |
| Tumeric Extract | 30.69 | Curcumin | 36.58 |
| Green Tea extract | 30.69 | ECGC (50%) | 18.29 |
| N-Acetylcysteine | 12.8 | N-Acetylcysteine | 15.26 |
| Vitamin B₆ | 3.12 | P5P or Pyridoxamine | 3.72 |
| R-α-Lipoic Acid | 7.68 | R-α-Lipoic Acid | 9.15 |
| Vitamin B₁ | 2.55 | Benofotiamine or | 3.04 |
| | | Thiamine pyrophosphate | |
| Vitamin E Succinate | 9.9 | Tocopherol succinate | 11.80 |
| Vitamin B₁₂ source | 0.615 | Hydroxocobalamin (1%) | 0.0073 |
| Folic acid/Folate source | 0.2 | Folic Acid (10%) | 0.024 |
| Piper nigrum extract | 0.258 | Piperine (98%) | 0.30 |
| Vitamin C | 1.54 | Ascorbic Acid or | 1.84 |
| | | Dehydroascorbic acid | |

*Other forms may be used. For example, hydroxocobalamin is an exemplary form of Vitamin B₁₂, but other active forms of Vitamin B₁₂ may be used.
**For example, Vitamin B₁₂, are often supplied as 1% active ingredient

The percent gross weight for each ingredient in the cocktail can be determined by scaling up the elemental or therapeutic levels for each ingredient by its total weight as provided by the raw material suppliers. For example, if the anticipated therapeutic level of EGCG is 100 mg and the green tea extract used is 50% EGCG, then the gross weight of the green tea extract would be 200 mg.

Standardization of the content of all herbal products (tumeric, Piper longum or Piper nigrum, and green tea) can be confirmed by certificate of analysis from the supplier and also by assay by an independent laboratory of the herbal products.

The various components can be obtained from the following manufacturers: turmeric and green tea from USA NutraSource (City of Industry, Calif.); black pepper from Sabinsa Corporation (Piscataway, N.J.); benfotiamine (B₁), pyridoxamine (B₆), hydroxycobalamin (B₁₂) and N-acetylcysteine from DNP International (Santa Fe Springs, Calif.); α-Lipoic acid, vitamin B₁₂, folic acid, and vitamin E from Stauber Ingredients (Fullerton, Calif.); and vitamin C in the form of ascorbic acid and dehydroascorbic acid from Harmony Concepts (Eugene, Oreg.). Of course, many ingredients are available from other suppliers as well and there is no limitation with respect to the source. Components should have high purity, preferably have a reliable analysis of the active ingredient, and be suitable for consumption.

The cocktails can be administered in any manner consistent with effective treatment of the diseases, disorders or conditions disclosed herein, or with reduction in the prevalence of one or more physiological markers associated with these diseases, disorders or conditions. For example, the cocktail can be administered one, two, three, four, five, six or more times daily; once every two, three, four, five or six days; once every week, every two weeks, every three weeks, or every four weeks; once per month, or at irregular intervals. The daily dosage can be provided in a single daily dose or multiple unit dosages taken during the day, either on a fixed or irregular schedule. The dosage form can be a liquid, for example a drink, or a solid, for example a tablet or capsule to be swallowed or a powder to be mixed with food or drink for consumption.

Uses

In some embodiments, the invention provides uses of the cocktails disclosed herein for treating a cognitive or neurological disorder. The treating can occur, for example, by reducing the prevalence of a physiological marker of AD, such as hyperphosphorylation of tau protein, for example abnormal phosphorylation at threonine 231, or an Aβ plaque precursor moiety.

In some embodiments, the invention provides uses of the cocktails disclosed herein in the preparation of a medicament for the treatment of a cognitive or neurological disorder. The invention also provides uses of a cocktail disclosed herein for reducing the prevalence of phosphorylation of tau protein at threonine 231 or an Aβ plaque precursor moiety.

According to these uses, the cocktail can be administered at any dosage level and via any regimen and route of administration disclosed herein, or using the dosages, regimens and/or routes of administration required to obtain the claimed effect. Furthermore, the cocktails according to these uses can comprise any combination of ingredients disclosed herein consistent with effective treatment of the cognitive or neurological disorder.

In addition to reducing the prevalence of physiological markers of AD and other cognitive or neurological disorders, the compositions of the present invention can also decrease, reverse or prevent several cellular-level markers and/or chemical processes that have been identified that either contribute to the development of neurological or cognitive deficiencies, particularly AD, or are present in higher amounts in individuals diagnosed with AD. In addition to reducing the prevalence of Aβ plaque precursor moieties and tau hyperphosphorylation, the compositions and methods of the present invention have a beneficial impact on one or more of at least four major biochemical phenomena or pathways: inflammation, oxidative stress, glycation/dysinsulinemia, and platelet function. These are referred to herein as AD Factors. However, these factors are not limited to Alzheimer's and are found in various other neurological and cognitive disorders as well. Several active compounds can be used to address these AD Factors. The use of a cocktail or mixture of these active compounds to prevent, slow or reverse the progression of Alzheimer's Disease, Parkinsons, ALS, mild cognitive impairment, and other types of dementia or neurological deterioration can address factors associated with the etiology or progression of these diseases.

The AD factors mentioned above can cause neuronal damage or neuronal death. For example, oxidative stress can cause neuronal damage. By administering a cocktail containing an antioxidant or component that reduces oxidative stress, neuronal damage can be reduced, prevented or possibly repaired. Importantly, oxidative stress can result not only in neuronal damage, but also damage to other cells and cellular systems as well. Use of an antioxidant is one way to reduce cellular damage in general.

The four pathways and their associated mechanisms, markers and factors are also set forth in Table 2. The compositions also beneficially affect a key marker, homocysteine levels, that is an important contributor to the development or progression of AD. Several naturally occurring compounds or groups of compounds have been shown to decrease, reverse or prevent these phenomena from occurring. The compositions and methods of the present invention address several of the different mechanisms that contribute to the onset or progression of AD, as well as other neurological deficiencies and diseases. Furthermore, the combination creates an environment where it is difficult for beta-amyloid plaques to either develop or deposit.

TABLE 2
AD Associated Mechanisms, Markers and Factors

| Oxidative Stress | Glycation | Inflammation | Platelet Function |
|---|---|---|---|
| Mitochondrial | Matrix | Tau and β amyloid | Secretion of Aβ |
| dysfunction | metalloproteinases | | |
| | (MMP) production | | |
| | | MMP excitation | Glutamate |
| Glutamate transport | Oxidation | Homocysteine | Inflammation |
| Beta amyloid | Inflammation | Heavy metals | PAF |
| Induced nitric oxide | β-Amyloid toxicity | | |
| synthase | | | |
| | Cognition | Mitochonrial | Aggregation |
| | | dysfunction | |
| Heavy Metals | Mitochondrial | TNF-β, NF-KB | +Capsase 3 |
| | dysfunction | | |
| Ubiquitin- | Advanced glycation | Platelet activation | |
| Proteosome. | end products | factor | |
| Heat shock protein | Pentosidine, N^{ε}- | | |
| | carboxymethyllysine | | |
| | (CML) | | |
| Platelet activation | | | |
| factor | | | |
| Cognition | | | |
| TBARS | | | |
| Malondialdehye | | | |
| 4-Hydroxnonenal | | | |

Table 3 lists the AD Factors and how components used in the cocktail of the invention address each. Several of these compounds address more than one of these factors. In addition, several of the ingredients have been shown to exhibit anticholinesterase activity. There are no known maximum daily dosage levels for many of these compounds, and many are not toxic unless, consumed in very high quantities. All are generally recognized to be safe for daily consumption.

TABLE 3
Medical Food Cocktail Ingredients and Disease Processes Targeted

| | Effect on Biochemical Processes | | | |
|---|---|---|---|---|
| Cocktail | Oxidative | | | Platelet |
| Ingredient | Stress | Inflammation | Glycation | Function |
| Curcumin | * | * | * | * |
| Piperine | * | * | * | * |
| Epigallocatechin- | * | * | | * |
| 3-gallate | | | | |
| (EGCG) | | | | |
| α-Lipoic Acid | * | * | | * |
| N-Acetylcysteine | * | * | * | |
| B₁ | | | * | |
| B₆ | * | * | * | * |
| B₁₂ | * | * | | |
| Folic Acid/Folate | * | | | |
| Vitamin C | * | * | | |
| Vitamin E | * | * | | * |

The following discusses four major biochemical phenomena or pathways that are implicated in the etiology of AD: inflammation, oxidative stress, glycation/dysinsulinemia, and platelet function.

Inflammation—Chronic inflammation has been observed to damage host tissue, and brain neurons are particularly vulnerable. Inflammatory mediators can be produced and elevated in affected regions in the brains of individuals with AD. Non-immune mediated chronic inflammatory responses in brain parenchyma, which can occur in response to the production of Aβ peptides, are believed to be involved in AD progression. Accordingly, the present invention provides treatment by reducing the production of, e.g., Aβ peptides, which provide at least one mechanism for inflammation, as well as treating inflammation that may occur through other mechanisms. Neurodegenerative plaque formation in AD is characterized by the up-regulation of interleukin (IL)-1 and IL-6, and this up-regulation can play a role in the pathogenesis of AD. Advanced glycation end products have been shown to exert an inflammatory effect as well. In some embodiments, the compositions and methods of the invention use naturally occurring compounds which individually act to slow or halt the chronic inflammatory-like process that occurs in the early pathological cascade of AD. Markers of inflammatory response include serum alpha (1) anti-chymotrypsin, nuclear factor-kappaBeta (NFKB), high sensitivity C-reactive protein, platelet activation factor, transforming growth factor beta, tumor necrosis factor (TNF)-alpha and inflammatory cytokine production in general. An inflammatory cascade precipitated by the formation of Aβ plaques in the brain is thought to be a prime cause of neuronal death. The inflammatory marker C-reactive protein and microglial inflammatory markers, such as the inflammatory cytokines IL-1β and IL-6 and the inflammatory proteins nitric oxide synthase-2 (NOS2) and TNF-alpha, are all up-regulated in tissue from Alzheimer's patients. C-reactive protein-like inflammation has been demonstrated in both the senile protein plaques (polymorphous beta-amyloid protein deposits found in the brain in Alzheimer disease and normal aging) and neurofibrillary tangles of Alzheimer's victims. Chronic inflammation may also be responsible for the degeneration of the hippocampus, a particularly vulnerable part of the brain. In some embodiments, compositions that have a beneficial impact on inflammation, such as those comprising naturally occurring compounds such as phytochemicals, can contribute to the prevention of AD and slowing its progression, especially because many of these processes are measurable long before clinical symptoms appear.

Oxidative Stress—Oxidative stress is caused by an imbalance between the production of reactive oxygen species (many of which are free radicals) and a biological system's ability to readily detoxify the reactive intermediates or easily repair the resulting damage. Alzheimer's patients also exhibit high serum levels of markers of oxidative stress and low plasma levels of antioxidants and free radical scavengers. Like inflammation, oxidative stress can play a role in the development and progression of most chronic degenerative diseases, including AD. Alzheimer's-diseased brains are characterized by excessive Aβ deposition and by extensive oxidative stress. There are several sources of oxidative stress, including advanced glycation end products, microglial activation and the sequelae of Aβ. Membrane permeable antioxidants prevent the up-regulation of induced nitric oxide synthase (iNOS), and some can be viewed both as antioxidants and as anti-inflammatory drugs. The destructive free radicals produced by oxidative stress can damage sensitive neurons. Metals such as iron, copper, zinc, and aluminum exacerbate the production of free radicals, as does the presence of Aβ plaques, creating a vicious cycle of neuronal damage. Accordingly, the present invention provides treatment by reducing the production, e.g., of Aβ peptides that provide at least one mechanism for oxidative stress, as well as treating oxidative stress that may occur through other mechanisms. Nutritional antioxidants can block or reduce neuronal death. Compositions of the present invention, which can include, for example, antioxidants, can contribute to preventing and/or slowing AD. Of particular interest are combinations of antioxidants that have complementary or synergistic activity, or that quench several types of reactive oxygen species.

Glycation/Dysinsulinemia—Glycation is the non-enzyme-mediated, generally haphazard reaction of protein or lipid molecules with sugars in a way that interferes with the activity of the protein or lipid. Glycation is the first step in the production of advanced glycation end-products (AGEs), which are a major cause of the physical manifestations of aging and damage to tissue elasticity. Extracellular AGEs can accumulate in the Aβ plaques of Alzheimer's patients, causing further oxidative stress on the surrounding neural tissue. Reducing Aβ plaques reduces accumulation of AGEs. AGEs are also found in the serum and cerebral spinal fluid of Alzheimer's patients. An increasing percentage of adults and children are overweight, and obesity often causes dysinsulinemia, which can lead to increased glycation of proteins. The incidence of non-insulin dependent diabetes mellitus (NIDDM) is increasing, even in people within normal body mass indices (BMIs). This trend, coupled with the potential effects of glycation on all types of dementia, is of concern. Glycoxidative (glycation+oxidation) stress creates a cascade of events leading to neurodegeneration, such as that found in AD. The accumulation of AGEs explains neuro-pathological and biochemical events such as protein cross linking, free radical damage, neuronal apoptosis and glial activation, all of which are features of AD. Several markers of glycoxidative stress have been identified. Examples of these markers are pentosidine, N(epsilon)-(carboxymethyl)lysine (CML), fructosamine, malondialdehyde (MDA), and 4-hydroxy-2-noneal (HNE). The compositions of the present invention, which can include, for example, AGE inhibitors, can slow, halt or reverse glycoxidative effects on AD.

Platelet Function—Platelets are a source of beta-amyloid precursor protein. Increased platelet activation, abnormal platelet function and increased circulating beta-amyloid have been observed in AD. Activated platelets are a source of Aβ peptides, and beta-amyloid tends to aggregate platelets and support their adhesion. Accordingly, the present invention provides treatment by reducing the production, e.g., of Aβ peptides that provide at least one mechanism for platelet activation, as well as treating platelet activation that may occur through other mechanisms. Non-steroidal anti-inflammatory drugs (NSAIDs) can reduce the inflammatory response of microglial cells. A significant correlation exists between platelet activating factor (PAF) binding and degree of cognitive impairment in Alzheimer's patients. Similarly, neurons pretreated with PAF antagonists were resistant to damage by Aβ and also exhibited a reduced activation of caspase-3, a marker of apoptosis (i.e., programmed cell death). Compositions of the present invention, which can include, for example, ingredients that have anti-inflammatory effects and/or that normalize platelet function, can be beneficial as therapeutic options in AD.

Homocysteine—Homocysteine presence or absence is believed to be a marker of, and/or a risk factor for, both stroke and cardiovascular disease. It has been estimated that exceeding normal levels (5-15 micromol/L) by as little as 5 micromol/L increases the risk of coronary artery disease by 60 percent in men and 80 percent in women. High homocysteine levels are also a risk factor for Alzheimer's disease. Individuals with a blood plasma homocysteine level above 14 micromol/L have been found to have nearly twice the risk of developing Alzheimer's disease as do people with lower levels. A 5 micromol/L increase in homocysteine level has been found to correspond to a 40 percent increased risk of developing Alzheimer's disease. Also, this damage can be halted and even reversed by repair of nerve cell DNA damage in the brain.

S-adenosylmethionine (SAMe), a biomolecule made from an amino acid molecule and ATP, is a substance that occurs naturally in the body. It is known to play a role in 35-40 biochemical reactions. In most people, the body can make all the SAMe it needs, but some patients with depression and other psychological conditions have been found to have lower levels of the compound, as well as lower levels of folate and vitamin B₁₂. Each of these substances plays a part in the metabolic process of “methyl donation,” or “methylation,” a process in which a methyl group is attached to a protein or lipid molecule. These methylation reactions are involved in the production of the neurotransmitters serotonin and dopamine in the brain as well as the activation of enzymes that help repair joints and the liver. There is evidence that serotonin is a factor in migraine and is involved in the so called “rebound effect,” because of its vasoconstricting effect at elevated levels and subsequent vasodilation as levels decrease. Folate deficiency also appears to reduce brain serotonin and contribute to depression in individuals. Folic acid supplementation can contribute to the achievement of an appropriate balance between serotonin generation and breakdown, which can lead to a decrease in the incidence of depression as well as a minimization of the cycling between vasodilation and vasoconstriction caused by fluctuations in serotonin levels.

In contrast to compositions that treat only the four AD Factors discussed above, the cocktails and methods disclosed herein are more targeted to physiological and cognitive abnormalities associated with neurological or cognitive disorders, because they can reduce the prevalence of tau protein hyperphosphorylation and/or Aβ plaque precursor moieties associated with neurological or cognitive disorders. Thus, the present invention provides for a directed approach focused on such biochemical causative factors of neurological and cognitive disorders. Reducing the prevalence of tau protein hyperphosphorylation and/or Aβ plaque precursor moieties can lead to a reduction in the susceptibility of neuronal cells and tissue to oxidative stress, glycation, inflammation and platelet aggregation, and ultimately cell death. Thus, while many of the components described herein can help to reduce the effects of one or more factors brought about by, e.g., the presence of tau hyperphosphorylation and/or Aβ plaque precursor moieties—i.e., oxidative stress, glycation, inflammation and platelet aggregation—a cocktail according to embodiments of the invention reduce the prevalence of the hyperphosphorylation and plaques themselves.

Furthermore, the deleterious effects of tau hyperphosphorylation on, for example, microtubule function, and the associated neuronal cell damage and/or death, can occur independently of the effects of oxidative stress, glycation, inflammation and platelet aggregation. Thus, therapies that only target these four AD Factors would not be expected to have an effect on microtubule dysfunction arising from tau hyperphosphorylation. In contrast, the cocktails and methods disclosed herein have been demonstrated to reduce tau phosphorylation, and therefore can be effective in reducing the prevalence of microtubule dysfunction and the associated pathologies.

As a result, the cocktails and methods disclosed herein enable the treatment of patient populations that were previously untreatable. An example of such a patient population includes those with a genetic predisposition to developing AD. AD has been shown to have a genetic component in many cases. For example, patients who express the E4 variant of the Apo E gene are three to eight times more likely to develop AD than people with other Apo E variants, such as E2 and E3. In addition, certain genetic mutations on chromosomes 1, 14 and 21 are associated with early-onset AD. For people with such genetic predispositions to develop AD, the pathophysiological effects of AD, including neuronal cell death due to tau hyperphosphorylation, may occur even if levels of oxidative stress, glycation, inflammation and platelet aggregation are maintained within normal ranges, especially in patients whose genetic predisposition to AD is strong. Accordingly, therapies that target only oxidative stress, glycation, inflammation and/or platelet aggregation would likely prove unsuccessful in bringing about long-term improvement in these patients. In contrast, treatment with therapies that have been shown to reduce tau hyperphosphorylation, such as the cocktails and methods disclosed herein, can be effective in providing long-term benefit to these patients, as well as other AD patients, regardless of their genetic predisposition.

Thus, the cocktails and methods can be used to treat a patient population—e.g., those with a strong genetic predisposition to developing tau hyperphosphorylation—for which no effective treatment was known prior to the cocktails and methods disclosed herein. These patients can be effectively treated regardless of whether their levels of oxidative stress, glycation, inflammation and/or platelet aggregation are elevated or within normal ranges. Stated differently, hyperphosphorylation and Aβ plaque formation can be distinct biochemical mechanisms that give rise to neudegenerative diseases and cognitive decline. Individuals susceptible to hyperphosphorylation and Aβ plaque formation can show a high propensity to develop neurological disease regardless of health with respect to of oxidative stress, glycation, inflammation and/or platelet aggregation. Thus, the present invention provides a method for treating disease not based on models treating only of oxidative stress, glycation, inflammation and/or platelet aggregation.

Curcumin is a polyphenol that comprises the active component of the plant/spice referred to as turmeric (Curcuma longa). The root and rhizome of turmeric have been used medicinally. The plant extract is standardized to 90-95% curcumin or curcuminoids.

Curcumin is a strong antioxidant, is a potent inhibitor of lipid peroxidation and has several anti-inflammatory effects. For example, curcumin is thought to bring about decreased histamine levels, increased natural cortisone production by the adrenals, and modified synthesis of specific interleukins, cytokines, leukotrienes and eicosanoids in general. Curcumin can modulate many inflammatory markers, such as TNF-a and NFKB. It also provides hepatoprotective benefits against a number of toxic compounds. Curcumin also demonstrates anti-platelet effects, which may protect against beta amyloid-induced platelet aggregation and platelet adhesions. It also has anti-glycation effects and can decrease levels of platelet-activating factor (PAF), thus disrupting normal platelet function. Curcumin protects normal human umbilical vein endothelial cells from Aβ. In studies on mice, low doses of curcumin significantly lowered levels of oxidized proteins and IL-1beta in mice brains. It also has been shown to suppress Aβ-induced cognitive defects and oxidative damage. According to the present invention, compositions comprising low dose curcumin can decrease insoluble Aβ, soluble Aβ, and plaque burden by, for example, 43-50%.

In Alzheimer transgenic mice, dietary curcumin is associated with decreased levels of oxidized proteins and interleukin-1 beta. A suppression of microgliosis has also been observed. In addition, curcumin prevents the accumulation of advanced glycation endproducts in diabetic rats receiving dietary curcumin (200 mg/kg body weight) compared to control diabetic rats without curcumin. It also brings about a significant reduction in lipid peroxidation products (which are indicators of oxidative stress) in the curcumin fed rats.

Compositions according to the present invention comprising, for example, curcumin are effective in the prevention and treatment of Alzheimer's disease. According to the invention, curcumin contributes to the inhibition of both the formation and growth of beta-amyloid fibrils from Aβ in a dose-dependent manner. Curcumin also inhibits neuroglial proliferation in rats. In a neuroblastoma cell line, curcumin inhibited activation of the inflammatory marker nuclear factor kappa-beta (NFKB). Likewise, curcumin inhibits inflammation-related cyclooxygenase-2 gene expression in microglial cells. Curcumin also inhibits platelet activating factor (PAF) and platelet aggregation induced by platelet agonists. Curcumin acts as a metal chelator, thus helping reduce Aβ aggregation and toxicity, while suppressing damage from inflammation. Along this line, curcumin has been shown to chelate both cadmium and lead in rat brain homogenates, protecting against lipid peroxidation. Supplementation with turmeric reduces oxidative stress and attenuates the development of fatty streaks in rabbits fed a high cholesterol diet. Thus, curcumin as an individual component has beneficial effects against neuronal damage brought about by the presence of Aβ plaques and tau protein hyperphosphorylation, but when incorporated into a cocktail disclosed herein, curcumin can contribute to a reduction in the prevalence of tau protein hyperphosphorylation and/or Aβ plaque precursor moieties. As a component of the cocktail described herein, curcumin can be administered in daily dosages of, for example, from about 250 mg to about 10 grams, or from about 250 mg to about 5 grams, or from about 500 mg to about 5 g, or about 1000 mg.

Alpha lipoic acid (ALA), a disulfide, is an antioxidant that is both lipid- and water-soluble. It promotes synthesis of the endogenous antioxidant glutathione. ALA can enhance glucose uptake, inhibit glycosylation and alleviate peripheral neuropathies and associated nerve pain. ALA prevents AGE-induced increases in NFKB activation, thus protecting against endothelial dysfunction. ALA stabilizes cognitive function in elderly, beginning-stage Alzheimer patients. The potential effectiveness of alpha-lipoic acid (ALA) against cytotoxicity induced by Aβ peptide (30 microM) and hydrogen peroxide (H₂O₂) (100 microM) with the cellular 3-(4,5-dimethylthiazol-2-yl)-2,5-diphenyl-tetrazolium bromide (MTT) reduction and fluorescence dye propidium iodide assays in primary neurons of rat cerebral cortex has been investigated. It was found that treatment with ALA protected cortical neurons against cytotoxicity induced by Aβ or H₂O₂. (Zhang L, Xing G Q, Barker J L, Chang Y, Maric D, Ma W, Li B S, Rubinow D R. Alpha-lipoic acid protects rat cortical neurons against cell death induced by amyloid and hydrogen peroxide through the Akt signaling pathway. Neurosci Lett 2001; 312:125-8. As an individual compound, ALA can thus protect against effects brought on by the presence of Aβ. However, when incorporated into a cocktail disclosed herein, ALA can contribute to a reduction in the prevalence of tau protein hyperphosphorylation and/or Aβ plaque precursor moieties.

AGEs have been shown to induce lipid peroxidation in a neuronal cell line in a dose-dependent manner. Blocking the specific AGE-receptor RAGE can reduce the AGE-mediated formation of lipid peroxidation products. Similar effects have been shown following administration of several antioxidants, such as alpha-lipoic acid, N-acetylcysteine, 17 beta-estradiol and/or aminoguanidine. Extracellularly-administered alpha-lipoic acid reduces AGE-albumin-induced endothelial expression of vascular cell adhesion molecule-1 (VCAM-1) and monocyte binding to endothelium in vitro, and has also demonstrated significant antioxidant potential. As a component of the cocktail described herein, ALA can be administered in daily dosages of, for example, from about 50 mg to about 2 grams, or from about 100 mg to about 1 gram, or from about 150 mg to about 500 mg, or about 300 mg. For example, the R form of ALA can be administered.

N-Acetylcysteine (NAC) administration has been studied in patients who met National Institute of Neurological and Communicative Disorders and Stroke-Alzheimer's Disease and Related Disorders Association criteria for probable AD. NAC was administered in a double-blind fashion, and testing for efficacy was done after 3 and 6 months of treatment. NAC treatment achieved beneficial results on nearly every outcome measure, although significant differences were obtained only for a subset of cognitive tasks.

Oxidative stress may play a crucial role in age-related neurodegenerative disorders. The ability of the two antioxidants, ALA and NAC, to reverse the cognitive deficits found in the SAMP8 mouse has been examined. By 12 months of age, this strain of mouse develops elevated levels of Aβ and severe deficits in learning and memory. Twelve-month-old SAMP8 mice, in comparison with 4-month-old mice, had increased levels of protein carbonyls (an index of protein oxidation), increased readings in the thiobarbituric acid reactive species (TBARS) assay (an indicator of lipid peroxidation) and a decrease in the weakly immobilized/strongly immobilized (W/S) ratio of the protein-specific spin label MAL-6 (an index of oxidation-induced conformational changes in synaptosomal membrane proteins). Chronic administration of either ALA or NAC improved cognition of 12-month-old SAMP8 mice in both the T-maze footshock avoidance paradigm and the lever press appetitive task without inducing non-specific effects on motor activity, motivation to avoid shock, or body weight. These effects are believed to have occurred directly within the brain, as NAC crossed the blood-brain barrier and accumulated in the brain. Furthermore, treatment of 12-month-old SAMP8 mice with ALA reversed all three indexes of oxidative stress. These results support the hypothesis that oxidative stress can lead to cognitive dysfunction, and they also provide evidence for a therapeutic role for antioxidants. NAC has also been shown to antagonize N-methyl-D-aspartate (NMDA)-caused glutamergic excitation and associated neurotoxicity. Thus, NAC as an individual component has beneficial effects against neuronal damage brought about by the presence of Aβ plaques and tau protein hyperphosphorylation. However, when incorporated into a cocktail disclosed herein, NAC can contribute to a reduction in the prevalence of tau protein hyperphosphorylation and/or Aβ plaque precursor moieties. As a component of the cocktail described herein, NAC can be administered in daily dosages of, for example, from about 100 mg to about 2 grams, or from about 250 mg to about 1500 mg, or from about 250 mg to about 1000 mg, or about 500 mg.

Vitamins C and E are well known for their anti-oxidant properties. In a study of more than 4740 subjects in Cache County, Utah, use of vitamin C and E supplements was associated with a significant reduction in risk of Alzheimer's disease. Similar results were seen in the Honolulu-Asia aging study of 3385 elderly men, in which vitamin C and E supplementation was associated with a protective effect for vascular and mixed dementia. Dementia patients and Alzheimer's patients also exhibit lower plasma vitamin C concentrations than control subjects with no cognitive impairment. Vitamin E prevents increased protein oxidation, reactive oxygen species, and Aβ-induced neurotoxicity in a rat embryonic hippocampal neuronal culture. Thus, vitamins C and E as individual components have beneficial effects against neuronal damage brought about by the presence of Aβ plaques and tau protein hyperphosphorylation, but when incorporated into a cocktail disclosed herein, vitamins C and E can provide additional benefits by contributing to a reduction in the prevalence of tau protein hyperphosphorylation and/or Aβ plaque precursor moieties. As a component of the cocktail described herein, a daily dosage can include, for example, at least about 100 mg of vitamin C as ascorbic acid or dehydroascorbic acid, or from about 10 mg to about 2 g, or from about 20 mg to about 1 g, or about 50 mg. A daily dosage can include, for example, from about 100 to 1000 IU of vitamin E, or from about 100 to about 800 IU, or from about 200 to about 600 IU, or about 400 IU as a component of the cocktail described herein. For example, vitamin E can be administered in the form of D-alpha tocopherols, tocopheryls, tocopheryl succinate, or a combination of these and other vitamin E isomers.

L-Carnosine (b-alanyl-L-histidine) is a naturally occurring di-peptide of the amino acids alanine and histidine. It is found in brain, muscle and other innervated tissues. High concentrations of carnosine are present in long-lived cells such as neuronal tissues and may be an aging marker. Carnosine, a powerful antioxidant, is active against by-products and metabolites of reactions with reactive oxygen species, and it also has an anti-glycation effect. MDA (malondialdehyde), a marker of DNA damage from oxidative stress, is blocked by carnosine.

Carnosine prevents sugar aldehydes from reacting with the amino acids in protein molecules, and also reverses the process. Carnosine's protection against cross-linking and the formation of abnormal AGEs, and its ability to reduce or prevent cell damage caused by Aβ, provide anti-aging benefits. In an 8-week study using L-carnosine, children with autistic spectrum disorders showed statistically significant improvements on the Gilliam Autism Rating Scale (total score and the Behavior, Socialization, and Communication subscales) and the Receptive One-Word Picture Vocabulary test (all p<0.05). Improved trends were noted on other outcome measures. Thus, carnosine as an individual component has beneficial effects against neuronal damage brought about by the presence of Aβ plaques and tau protein hyperphosphorylation, but when incorporated into a cocktail disclosed herein, carnosine can contribute to a reduction in the prevalence of tau protein hyperphosphorylation and/or Aβ plaque precursor moieties. Although the mechanism of action of L-carnosine is not well understood, it may enhance neurologic function, perhaps in the enterorhinal or temporal cortex. As a component of the cocktail described herein, L-carnosine can be administered in daily dosages of, for example, at least about 100 mg.

Epigallocatechin-3-gallate (EGCG), a polyphenol commonly recovered from green tea extract, which is standardized to a minimum of 50% EGCG, is a potent anti-inflammatory and antioxidant compound. EGCG is believed to be involved in amyloid precursor protein (APP) secretion and protection against toxicity induced by Aβ. EGCG can decrease or prevent Aβ toxicity in P C12 cells. Green tea can improve age-related cognitive decline and confer neuroprotection in Alzheimer's disease models. Although initially ascribed to the antioxidant properties of green tea, the neuroprotective effects may be due to a wide spectrum of cellular signaling events targeting many disease processes.

In cultured hippocampal neurons exposed to Aβ for a 48-hour period, co-treatment of the cells with EGCG decreased the levels of malondiadehyde (a marker for glycation) and caspase C (a marker of abnormal platelet function) compared to controls with no EGCG. Cells treated with EGCG also exhibited increased survival compared to controls. Similarly, a water-based extract of green tea inhibited the aggregation of rabbit platelets in vitro. The investigators found that green tea was comparable to aspirin in preventing platelet aggregation. Finally, EGCG was shown to inhibit the inflammatory markers TNF-a and NFKB, as well as interleukin-1 proinflammatory signal transduction in cultured epithelial cells. It also appears that EGCG may protect against ischemic neuronal damage. While EGCG by itself can help control damage by exposure to Aβ, a cocktail as disclosed herein incorporating EGCG as one ingredient can prevent the formation of Aβ plaques and/or reduce their prevalence, which reduces the need for such control. As a component of the cocktail described herein, EGCG can be administered in daily dosages of, for example, from about 10 mg to about 3000 mg, or from about 10 mg to about 1500 mg, or from about 50 mg to about 1000 mg, or about 600 mg.

Complex vitamins (such as B₆, B₁₂ and folic acid/folate) can prevent or reduce homocysteine (HC) damage. Elevated HC levels induce direct neurotoxicity and potentiate Aβ and glutamate neurotoxicity. The B vitamins may both improve cognitive functioning and reduce the levels of biochemical markers for Alzheimer's disease processes. In cultured brain cells grown in media deficient in folic acid, the addition of methotrexate (a folic acid inhibitor) to the media rendered nerve cells more susceptible to death from Aβ. Likewise, in a mouse model of Alzheimer's a folic acid-deficient diet resulted in DNA damage and damage to the hippocampus. In patients from the Framingham Heart Study, low levels of plasma B₆ were correlated with high levels of the inflammatory marker C-reactive protein. In patients with mild cognitive impairment and increased homocysteine levels, treatment with a B₆-B₁₂-folic acid combination improved blood brain barrier function and appeared to stabilize cognitive status. In in vitro studies, vitamin B₁ inhibited formation of advanced glycation end products in bovine serum albumin, ribonuclease A, and human hemoglobin. Low B₁₂ and folate blood levels are associated with dementia. Vitamins B₆, folate and B₁₂ can reduce these elevated HC levels. Vitamin B₅ (pantothenic acid) is also necessary to form acetylcholine. Additionally, certain lesser-known metabolites or alternative forms of some of the B vitamins, such as B₁, B₆ and B₁₂, play important roles in AD beyond their identified uses for reduction of homocysteine. For example, the hydroxycobalamin form of B₁₂ has been found to scavenge NO radicals, which have been associated with neurodegeneration and migraines. The benfotiamine form of vitamin B₁, which is a form of vitamin B₁ that can be used in the cocktails and methods disclosed herein, is a fat-soluble form of vitamin B₁ and has demonstrated significant benefit against excessive glycation and advanced glycation endproducts (AGEs). Furthermore, folic acid compositions have been found to address inflammation, for example that caused by NO, as well as endothelial function. Nitrogen oxide (NO) synthase creates NO which causes inflammation in tissue. Thus, these vitamins as individual components have beneficial effects against neuronal damage brought about by the presence of Aβ plaques and tau protein hyperphosphorylation, but when incorporated into a cocktail disclosed herein, these vitamins can contribute to a reduction in the prevalence of tau protein hyperphosphorylation and/or Aβ plaque precursor moieties.

The beneficial properties of folic acid can also be enhanced by the concurrent use of certain B vitamins and antioxidants such as vitamin E, s-adenosylmethionine (SAMe) and coenzyme Q10 (CoQ10). Any form of the B vitamins can be used, for example pyridoxal-5-phosphate (P5P) and hydroxycobalamin. Addition of NO synthase inhibitors, such as amino-guanidine, L-carnitine, asymmetric arginine, and certain plant derived phytochemicals, can enhance the inflammation-reducing properties of folic acid. For example, a daily dosage can comprise 100 mcg to 10 mg, or from about 1 mg to about 10 mg, or from about 2 mg to about 8 mg, or about 5 mg folic acid. As a component of the cocktail described herein, a daily dosage can also comprise one or more of from about 100 mcg to 10 mg, or from about 100 mcg to about 5 mg, or from about 100 mcg to about 2.5 mg, or about 200 mcg, of the hydroxycobalamin, hydroxocobalamin, hydrocobalamin, adenosylcobalamin or methylcobalamin form of vitamin B₁₂; from about 1 to about 300 mg, or from about 10 to about 200 mg, or from about 25 to about 150 mg, or about 100 mg of B₆ (pyridoxal-5-phosphate or pyridoxamine); from about 10 to 500 mg, or from about 10 to 250 mg, or from about 10 to 200 mg, or about 100 mg of vitamin B₁; and from about 10 mg to about 1,000 mg, or from about 10 to 500 mg, or from about 10 to about 100 mg, or about 25 mg of riboflavin (vitamin B₂).

Folic acid or salts thereof, referred to as folates, along with vitamins B₆ and B₁₂, are required in metabolic pathways involving methionine, homocysteine, cystathionine, and cysteine. The term “folates,” as used herein, is meant to include, at a minimum, folacin (USP folic acid), naturally occurring folinic acid, 5-methyl tetrahydrofolate, and tetrahydrofolate as well as salts or metabolites of these compounds. It appears that folate, B₆ and B₁₂ are all necessary for normal metabolism. However, these three compounds each function in a different manner. Folic acid, even if available at normal levels, is consumed in the metabolic process and therefore must be constantly replenished by diet or supplements. However, B₆ and B₁₂ function as co-factors. While necessary for the respective metabolic process to proceed, they are each regenerated. Therefore, if they are present in normal amounts in serum, supplementation may not be necessary. B₁₂ in the form of 5′-deoxyadenosylcobalamin is an essential cofactor in the enzymatic conversion of methylmalonylCoA to succinylCoA. The remethylation of homocysteine (HC) to methionine, which is catalyzed by methionine synthase, requires folate in the form of methyltetrahydrofolate and B₁₂ in the form of methylcobalamin. HC is condensed with serine to form cystathionine (CT) in a reaction catalyzed by cystathionine beta-synthase, which requires B₆ (pyridoxal phosphate). CT is also hydrolyzed in another B₆-dependent reaction to cysteine and alpha-ketobutyrate. Homocysteine is a modified form of the amino acid methionine, and it is tightly regulated by enzymes which require folate. By impairing DNA repair mechanisms and inducing oxidative stress, homocysteine can cause the dysfunction or death of cells in the cardiovascular and nervous systems. Homocysteine appears to be present in many disease states. However, dietary folic acid stimulates homocysteine removal and may thereby protect cells against disease processes.

The principal biochemical function of folates is the mediation of one-carbon transfer reactions. 5-methyltetrahydrofolate donates a methyl group to homocysteine in the conversion of homocysteine to L-methionine. The enzyme that catalyzes this reaction is methionine synthase. Vitamin B₁₂ is a cofactor in the reaction. This reaction is important in the regulation of serum homocysteine levels. The L-methionine produced in the reaction can participate in protein synthesis and is also a major source for the synthesis of S-adenosyl-L-methionine (SAMe). The methyl group donated by 5-methyltetrahydrofolate to homocysteine in the formation of L-methionine is used by SAMe in a number of transmethylation reactions involving nucleic acids, phospholipids and proteins, as well as in the synthesis of epinephrine, melatonin, creatine and other molecules. Tetrahydrofolate is the folate-containing product of the methionine synthase reaction. 5-Methyltetrahydrofolate is generated by conversion of 5,10-methylenetetrahydrofolate into 5-methyltetrahydrofolate via the enzyme methylene-terahydrofolate reductase (MTHFR). 5,10-Methylenetetrahydrofolate is regenerated from tetrahydrofolate via the enzyme serine hydroxymethyltransferase, a reaction which, in addition to producing 5,10-methylenetetrahydrofolate, yields glycine.

In addition to its role in the metabolism of homocysteine, 5,10-methylenetetrahydrofolate supplies the one-carbon group for the methylation of deoxyuridylic acid to form the DNA precursor thymidylic acid. This reaction is catalyzed by thymidylate synthase and the folate product of the reaction is dihydrofolate. Dihydrofolate is converted to tetrahydrofolate via the enzyme dihydrofolate reductase.

Folates are also involved in reactions leading to de novo purine nucleotide synthesis, interconversion of serine and glycine, generation and utilization of formate, the metabolism of L-histidine to L-glutamic acid, the metabolism of dimethylglycine to sarcosine and the metabolism of sarcosine to glycine.

One of the natural folates, folinic acid, is used as a pharmaceutical agent. Folinic acid, which is also known as leucovorin, citrovorum factor or 5-formyltetrahydrofolate, is used as rescue therapy following high-dose methotrexate in the treatment of osteosarcoma. It is also used to diminish the toxicity of methotrexate. It is used in the treatment of megaloblastic anemia resulting from folate deficiency, and also in the prevention or treatment of the toxic side effects of trimetrexate and pyrimethamine. The combination of folinic acid and 5-fluorouracil has until recently been standard therapy for metastatic colorectal cancer. Folinic acid increases the affinity of fluorouracil for thymidylate synthase. Folinic acid is available as a calcium salt for parenteral or oral administration.

Folic acid is also called pteroylglutamic acid or PGA. Its full chemical name is N-[4-[[(2-amino-1,4-di-hydro-4-oxo-6-pteridinyl)methyl]amino]benzoyl]-L-glutamic acid. Older names for folic acid are vitamin Bg, folicin, vitamin Bc and vitamin M. Its molecular formula is C₁₉H₁₉N₇O₆ and its molecular weight is 441.40 daltons. Folic acid forms yellowish-orange crystals. The color is imparted by the pteridine ring of folic acid. Pteridine also imparts color to butterfly wings.

Folic acid has been prescribed as a nutritional supplement for many medical conditions associated with elevated homocysteine levels. Folic acid supplements appear to reverse the elevated homocysteine levels. However, the elevated homocysteine level may be a result of inadequate supply or excessive consumption of folic acid and not the cause of the disease. It is clinically beneficial in such instances to provide folic acid supplements because individuals with elevated homocysteine levels appear to be at increased risk for cardiovascular disease and stroke, and neurodegenerative disorders such as Alzheimer's and Parkinson's diseases as well as neural tube defects, spontaneous abortion, placental abruption, low birth weight, renal failure, rheumatoid arthritis, alcoholism, osteoporosis, neuropsychiatric disorders, non-insulin-dependent diabetes and complications of diabetes, fibromyalgia and chronic fatigue syndrome. Moderate elevations of HC might be associated with increased risk for vascular disease (Ueland et al. (1992) in Atherosclerotic Cardiovascular Disease, Hemostasis, and Endothelial Function (Francis, Jr., ed.), Marcel Dekker, Inc., New York, pp. 183-236). However, folic acid deficiencies have also been associated with periphereal vascular disease and coronary disease in individuals with normal homocysteine levels (Bunout, D. et al “Low Serum Folate but Normal Homocysteine Levels in Patients with Atheroslerotic Vascular Disease and Matched Healthy Controls”, Nutrition 2000, 16, p. 434-8), suggesting that folic acid may have a protective effect that extends beyond maintaining normal homocysteine levels. In addition, moderate hyperhomocysteinaemia has been shown to be frequently present in cases of stroke and to be independent of other stroke risk factors (Brattstrom et al. (1992) Eur. J. Clin. Invest. 22:214-221).

It is not clear if the various disease states are caused by elevated homocysteine levels or the elevated homocysteine levels are caused by other factors which are the primary cause of the disease state and result in elevated levels of homocysteine. For example, it is also known that folic acid supplements are useful where B₁₂ deficiencies exist, but where homocysteine levels may not be elevated. Individuals with B₁₂ deficiency can display neurologic disorders, typically relating to underlying anemia. However, supplementing diet with only folic acid is not medically recommended as these folic acid supplements may mask the underlying B₁₂ problem. U.S. Pat. No. 4,945,083, issued Jul. 31, 1990 to Jansen, entitled Safe Oral Folic Acid-Containing Vitamin Preparation, describes an oral vitamin preparation comprising the combination of 0.1-1.0 mg B₁₂ and 0.1-1.0 mg folate for the treatment or prevention of megaloblastic anemia.

Normal serum folate levels in healthy individuals are 2.5-20 ng/ml, with levels less than 2.5 ng/ml indicating the possibility of clinically significant deficiency. Like B₁₂ serum levels, however, serum folate levels are a relatively insensitive measure in that only 50-75% of patients with folate deficiency have levels less than 2.5 ng/ml, with most of the remaining 25-50% being in the 2.5-5.0 ng/ml range (Allen (1991), Cecil Textbook of Medicine, 19th Ed.).

A series of patents to Allen et al, (U.S. Pat. No. 5,563,126, U.S. Pat. No. 5,795,873, U.S. Pat. No. 6,207,651, U.S. Pat. No. 6,297,224 and U.S. Pat. No. 6,528,496)) teaches the use of oral compositions or a transdermal patch delivering a combination of B₁₂ and folate, or B₁₂, folate and B₆, in concentrations sufficient to reduce elevated homocysteine levels by treating either single or multiple deficiencies of B₁₂, folate, and B₆. The Allen non-prescription formulations include 0.3-10 mg CN-cobalamin (B₁₂) and 0.1-0.4 mg folate or 0.3-10 mg B₁₂, 0.1-0.4 mg folate, and 5-75 mg B₆. The Allen prescription formulations comprise between 0.3-10 mg CN-cobalamin (B₁₂) and 0.4-10.0 mg folate or 0.3-10 mg B₁₂, 0.4-1.0 mg folate, and 5-75 mg B₆.

Piperine, a component of the spice black pepper, increases the bioavailability of curcumin and epigallocatechin-3-gallate. Piperine also exhibits significant antioxidant activity of its own, as well as significant chemopreventative and immunomodulary effects. However, when incorporated into a cocktail disclosed herein, piperine can contribute to a reduction in the prevalence of tau protein hyperphosphorylation and/or Aβ plaque precursor moieties. As a component of the cocktail described herein, a daily dosage of piperine can contain, for example, at least about 2.5 mg of piperine, or from about 1 to about 100 mg, or from about 5 to about 20 mg, or about 10 mg of piperine. A preferred source is derived from Piper longum or Piper nigrum (black pepper or long pepper) and standardized as 90%+piperine.

Although the above components are shown to have some effect on cognitive development by reducing cellular damage, there is no prior evidence of these materials, alone or in combination, reducing the prevalence of physiological markers of AD, such as Aβ plaque precursor moieties and/or tau protein hyperphosphorylation, and/or reduce their prevalence in the scope and magnitude demonstrated here, and thus delay the onset, arrest or reverse the development of, or treat behavioral or physiological effects of AD. In fact, to date there is no effective treatment for AD. It has surprisingly been found that a combination of these ingredients dramatically treats AD.

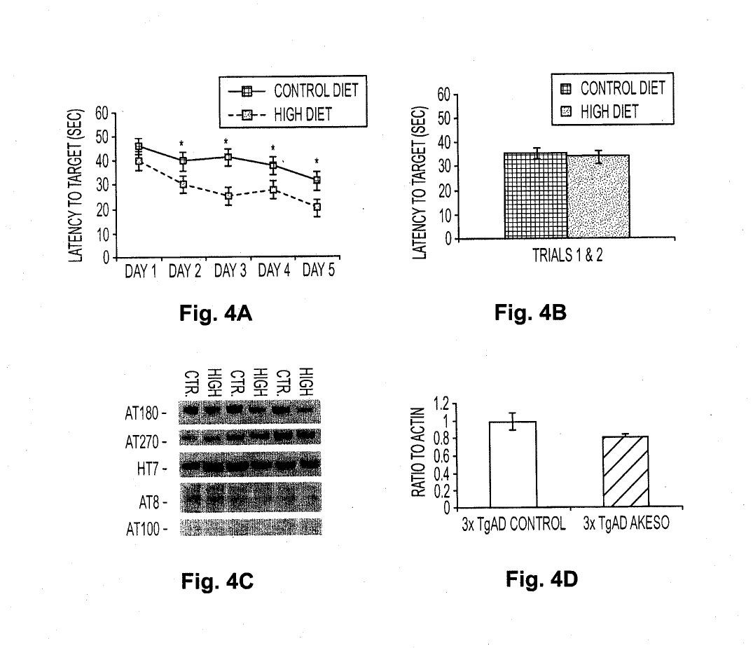

The inventive compositions have been shown to have beneficial effects on the levels of physiological markers of AD. For example, administration of the inventive compositions in two well-regarded mouse models of AD has been shown to result in marked decreases in the levels of, for example, soluble Aβ42, C99, and Aβ*56, all of which have been shown to contribute to the physiological progression of AD (see, e.g., FIGS. 4A-F, 5D). These results demonstrate that the inventive compositions are unexpectedly effective in reducing the levels of the physiological markers of AD, a vital step in slowing or reversing the progression of AD.

The compositions and methods of the present invention provide a surprising advance over previously-available AD therapies. Previously-available treatments were thought, at best, to affect only the cellular-level mechanisms associated with the progression of AD and other diseases and conditions associated with cognitive decline. Such treatments can perhaps delay the symptomatic progression of the disease, but they do nothing to reduce or eliminate the physiological pathologies that give rise to the symptoms of the disease to the scope and magnitude observed for the present invention. The compositions and methods of the present invention have been shown to prevent the formation, and/or reduce the prevalence, of Aβ plaques and tau tangles themselves, thus targeting the physiological markers that give rise to many of the cellular-level toxic mechanisms. Thus, while previously-existing treatments were thought to exert only an indirect effect on the progression of the disease, the compositions and methods disclosed herein have been demonstrated to have a direct effect on one or more of the root causes of AD and other cognitive and neurological disorders.

Furthermore, the compositions and methods also have been shown to decrease tau hyperphosphorylation. Efficacy in targeting this particular aspect of AD progression offers health care professions a new option in treating AD and other cognitive diseases. First, for patients in which it is more beneficial to treat the tau-centered effects rather than the Aβ-centered pathologies—for example, patients in which tau-based neuronal loss or other pathologies related to cytoskeletal disruption are more prevalent, or those in which neuronal pathologies are more prevalent in brain regions characterized by tau hyperphosphorylation—the compositions and methods provide a composition demonstrated to target this pathology. In addition, in diseases such as frontotemporal dementia, which is characterized by formation of tau tangles but not Aβ plaques, the compositions and methods have been demonstrated to be particularly well-suited for treatment.

Accordingly, the inventive compositions provide a surprising an unexpected advance in the art, as no effective treatment for AD existed prior to the present invention, and no treatment has been shown to reduce the prevalence of physiological markers such as tau hyperphosphorylation and Aβ plaque precursor moieties, and/or to reduce them in the scope and magnitude demonstrated here. Furthermore, the inventive compositions are especially beneficial because they include all-natural ingredients that are generally well-tolerated in patients.

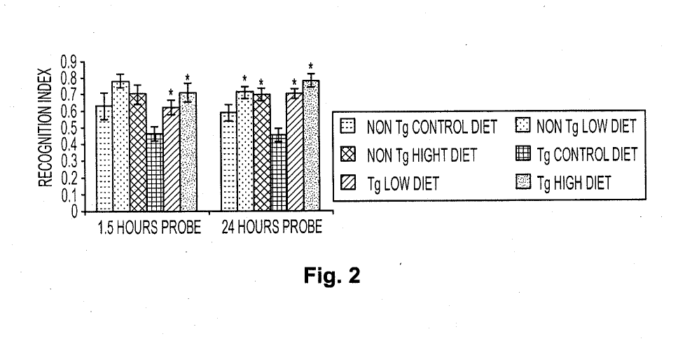

The compositions according to the present invention have been shown to prevent deterioration in cognitive performance on hippocampal and cortical dependent tasks in two different mouse models of Alzheimer's disease. Transgenic mice that were predisposed to develop Aβ plaques performed markedly worse than non-transgenic mice on a series of well-regarded cognitive tasks. However, transgenic mice that were administered the inventive compositions were statistically indistinguishable from non-transgenic mice in their performance on the same cognitive tasks (see, e.g., FIGS. 2A-F, 3, 5A). These results provide powerful evidence that the inventive compositions are effective in treating the cognitive deficits that arise in AD.

It is preferred that these compositions be delivered orally and the components be prepared for ingestion in a manner that makes the composition available in therapeutically effective amounts. As such, they may be prepared as water soluble compositions, delivered in liquid form, lyophilized, encapsulated, or formulated in a manner suitable for time release, delayed release or enteric delivery, or any manner typically used for orally-delivered pharmaceuticals, nutraceuticals or vitamins, or combined with foods or other normally-ingested products. However, the invention is not limited to oral delivery as the compositions set forth herein may also be delivered by nasal spray, inhalation techniques, transdermally, transmucossally, by suppository, injected or by intravenous methods.

The following examples are provided in order to better enable one of ordinary skill in the art to make and use the disclosed compositions and methods, and are not intended to limit the scope of the invention in any way.

### EXAMPLES

### Example 1

### Therapeutic Evaluation in Tg2576 Mice

### Example 1a

### Cognitive Evaluation

We theorized that a combination therapy comprising a variety of antioxidants and vitamins would prove most efficacious in the treatment of AD in humans. To support this we tested the following combination of nutraceuticals in two well-regarded and utilized mouse models of AD:

TABLE 4
Components of cocktail concentrate as well as low and
high concentration diets added to AIN-17 rodent chow

| | | High | Low |
|---|---|---|---|
| | | Concentration | Concentration |
| | Concentration | Diet (mg/kg | Diet (mg/kg |
| | (%) | chow) | chow) |
| Curcumin extract | 30.69 | 202.55 | 67.52 |
| Green Tea extract | 30.69 | 202.55 | 67.52 |
| (contains 50% EGCG) | | | |
| N-Acetylcysteine | 12.8 | 84.48 | 28.16 |
| Vitamin B₆ | 3.12 | 20.59 | 6.86 |
| R-α-Lipoic Acid | 7.68 | 50.69 | 16.90 |
| Vitamin B₁ | 2.55 | 16.83 | 5.61 |
| (benfotiamine) | | | |
| Vitamin E Succinate | 9.9 | 65.34 | 21.78 |
| Vitamin B₁₂ source | 0.615 | 4.06 | 1.35 |
| (contains 1% B₁₂) | | | |
| Folic acid source | 0.2 | 1.32 | 0.44 |
| (contains 10% | | | |
| folic acid) | | | |
| Bioperine ® | 0.258 | 1.70 | 0.57 |
| (contains | | | |
| 98% piperine) | | | |
| Vitamin C | 1.54 | 10.16 | 3.39 |
| Total | | 660.27 | 220.10 |

The high concentration diet and low concentration diet cocktails were prepared to provide about 660 and about 220 mg cocktail per 1 kg rodent chow, respectively, as noted above. Green tea extract, Bioperine®, and the folic acid and vitamin B₁₂ sources each provided a percentage of the respective active ingredient. Green tea provided 50% EGCG; Bioperine® provided 98% piperine; the folic acid source provided 10% folic acid; and the vitamin B₁₂ source provided 1% vitamin B₁₂. Factoring in these percentages, and assuming an average mouse weight of 20 g and an average daily consumption of 5 g chow per mouse, gives rise to the following daily intakes for each active ingredient and for the cocktail as a whole:

TABLE 5
Daily intake of cocktail components on high and low concentration diets

| | Active | High | Consumption | Low | Consumption |
|---|---|---|---|---|---|
| | Ingredient | Concentration | per mouse - | Concentration | per mouse - |
| | Concentration | Diet (mg/kg | High (mg/kg | Diet (mg/kg | Low (mg/kg |
| | (%) | chow) | body wt/day) | chow) | body wt/day) |
| Curcumin | 36.58 | 202.55 | 50.64 | 67.52 | 16.88 |
| EGCG | 18.29 | 101.28 | 25.32 | 33.76 | 8.44 |
| N-Acetylcysteine | 15.26 | 84.48 | 21.12 | 28.16 | 7.04 |
| Vitamin B₆ | 3.72 | 20.59 | 5.15 | 6.86 | 1.72 |
| R-α-Lipoic Acid | 9.15 | 50.69 | 12.67 | 16.90 | 4.23 |
| Vitamin B₁ | 3.04 | 16.83 | 4.21 | 5.61 | 1.40 |
| (benfotiamine) | | | | | |
| Vitamin E | 11.80 | 65.34 | 16.34 | 21.78 | 5.45 |
| Succinate | | | | | |
| Vitamin B₁₂ | 0.0073 | 0.041 | 0.01 | 0.014 | 0.003 |
| Folic acid | 0.024 | 0.13 | 0.033 | 0.044 | 0.011 |
| Piperine | 0.30 | 1.67 | 0.42 | 0.56 | 0.14 |
| Vitamin C | 1.84 | 10.16 | 2.54 | 3.39 | 0.85 |
| Total | 100.00 | 553.75 | 138.44 | 184.60 | 46.15 |

The diets were fed to Tg2576 and non-transgenic (nonTg) control mice at 6 months of age. After 6 months of treatment mice were tested for cognition on hippocampal and cortical dependent tasks. The first task used was the widely utilized Morris water maze (FIG. 2A-F). This task tests spatial memory and consists of 4 daily trials over 7 days, in which mice must learn the location of a hidden platform. As mice acquire the task they should reach the platform quicker, indicating improved spatial memory. Acquisition curves demonstrated that 12-month-old Tg2576 mice were severely cognitively impaired compared to age-matched nonTg mice. (FIG. 2A.) However, Tg2576 mice treated with either the low or high concentration diet acquired the task significantly better than untreated Tg2576 mice, and were statistically indistinguishable from nonTg mice. These results show that the cocktail-containing diets prevent cognitive deficits associated with development of AD pathology on hippocampal spatial acquisition. NonTg mice treated with the cocktail-containing diet acquired the task to a similar degree as the untreated nonTg mice. In order to ensure that all mice were starting off at the same level we averaged the first two trials of the first day of training for each group (FIG. 2B). All groups were statistically insignificant from one another, showing that all groups initially performed equally, but then learned the task at different rates. Spatial reference memory probe trials were conducted at 1.5-h and 24-h after the last training trial to examine short and long-term memory, respectively. Consistent with the acquisition curves, Tg2576 mice showed impaired latencies to cross the platform location as compared to nonTg mice at both the 1.5 and 24 h probes (FIG. 2C). Tg2576 mice treated with cocktail-containing diet performed at untreated nonTg levels, thus completely preventing the deficits seen in the untreated Tg2576 mice. Similar results were seen in the number of platform crosses (FIG. 2D), time spent in the target quadrant (FIG. 2E), and time spent in the opposite quadrant (FIG. 2F). These results show complete prevention of cognitive deficits seen in Tg2576 mice, which arise due to AD-like pathology, by treatment with either a low or high concentration diet.

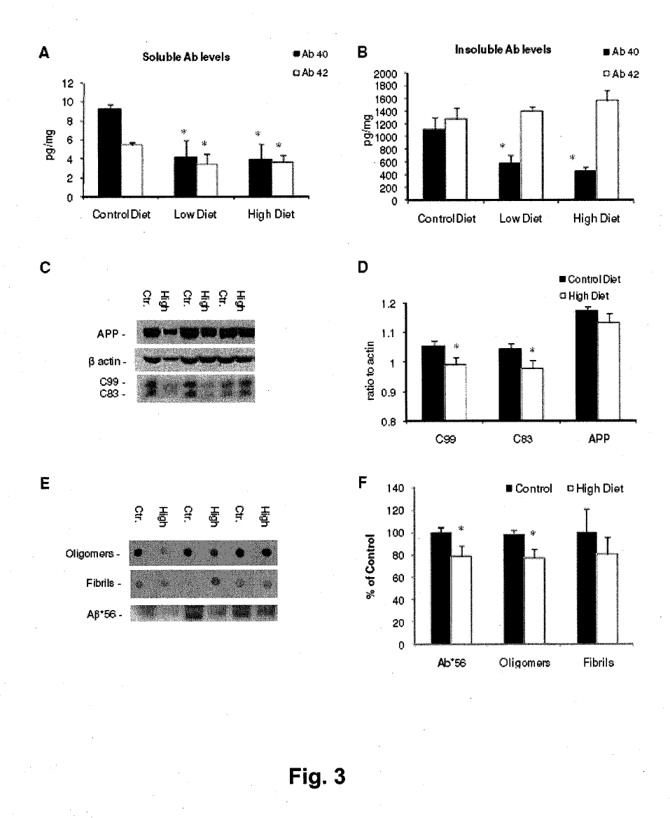

Finally, we evaluated cocktail diet-treated and untreated Tg2576 and nonTg mice in performance of the cortex-dependent contextual task, novel object recognition, which relies on the animals' preference to explore a novel object over a familiar object. After familiarization with the object, mice were reintroduced to the familiar, as well as a novel, object 1.5 and 24 h later. The ability of mice to remember which object they had seen before was then assessed. Tg2576 mice explored at chance level, indicating that they were not discriminating between the 2 objects suggesting that they could not recall the familiar object. NonTg mice spent significantly more time with the novel object, showing that they could recall the familiar object (FIG. 3). Treatment with cocktail-containing diet significantly improved nonTg performance at the 24-hour probe, suggesting that treated nonTg mice had improved cortical dependent memory compared to untreated nonTg mice. Notably, treatment of Tg2576 fully restored performance to nonTg levels at both 1.5- and 24-hour probe trials. These results show that either low or high concentration diet fully prevented cortical cognitive deficits, which arise due to AD-like pathology in the Tg2576 mice.

### Example 1b

### Physiological Evaluation

To assess the disease modifying effects of the cocktail-containing diet, we looked at brain pathology in untreated Tg2576 mice versus Tg2576 mice treated with high and low concentration diets. Levels of soluble Aβ40 and 42 were significantly reduced for both treatment groups (i.e., those treated with low and high concentration diets (FIG. 4A); levels of insoluble Aβ40 were also significantly reduced (FIG. 4B). Soluble Aβ42 is the form most often associated with disease states including AD. Given these striking reductions in Aβ levels we assessed levels of its precursors C99 and APP. APP is cleaved by α-secretase to form C83 and an n-terminal fragment, whereas β-secretase cleaves APP into C99 and an n-terminal fragment. C99 and C83 are both cleaved by gamma-secretase. Cleavage of C99 by gamma-secretase results in Aβ.

We found no changes in steady state levels of APP but significant decreases in C99, and also C83 (FIG. 4C, D). These results show that the cocktail-containing diet is disease modifying, exerting a direct effect on physiological markers for AD, in addition to being able to prevent cognitive deficits in Tg2576 mice.

A tremendous amount of recent evidence has highlighted the aggregation state of Aβ in being important to its pathological activities, rather than just its levels. Low molecular weight oligomeric Aβ species have been highlighted as the most toxic, and have been shown to be potent inhibitors of long-term potentiation (LTP), a form of synaptic plasticity thought to underlie memory, as well as proteasome function leading to the accumulation of other intracellular proteinaceous aggregates. As such, therapies that target the breakdown of these oligomers are of great interest. To assess levels of low molecular weight oligomeric Aβ species we used the conformation specific antibody All. Dot blot analysis showed a 50% reduction of these toxic soluble oligomers in the brains of Tg2576 animals treated with the high concentration diet (FIG. 4E). A soluble Aβ dodecamer designated Aβ*56 has been highlighted as an oligomeric species that causes memory deterioration and synaptic dysfunction. Analysis of this Aβ dodecamer revealed a 50% reduction in Tg2576 mice treated with the high concentration diet, compared to untreated mice (FIG. 4F). Such a therapy is of enormous potential, and is believed to be the only oral treatment to date shown to affect this crucial Aβ species.

### Example 2

### Therapeutic Evaluation in 3xTg-AD Mice

Given these extremely promising results we sought to validate the cocktail-containing diet by testing the high-concentration diet in a second mouse model of AD, the 3xTg-AD mouse model. The 3xTg-AD mice progressively develop Aβ and tau pathology, with a temporal- and regional-specific profile that closely mimics their development in the human AD brain. Despite equivalent expression of the human APP and human tau transgenes, Aβ deposition develops prior to the tangle pathology. Extracellular Aβ deposits manifest by 6 months of age in the cortex, and by 12 months are thioflavin S-positive and also positive for Congo red. Tau becomes mislocalized to the somatodendritic compartment at ˜6 months of age, and reactivity with conformational specific antibodies such as the mouse monoclonal antibody MC-1 is apparent by 10 months, followed shortly thereafter by immunoreactivity for phospho-specific tau markers at about 10-12 months of age. The tau pathology follows a hierarchal pattern, with MC1 immunoreactivity emerging first, followed by phospho-specific markers such as AT8 and AT180, and then lastly PHF. This closely mimics the pathology that occurs in the human brain.

3xTg-AD mice were 4 months of age at the beginning of treatment. Hippocampal dependent spatial memory was assessed five months later using the Morris water maze. In accordance with the Tg2576 mice we found that the combination diet treated 3xTg-AD mice performed significantly better than the untreated 3xTg-AD mice on every day of training except the first (FIG. 5A-B). This indicates that the combination diet treated mice learn the task at a faster rate than the untreated mice. In addition, administration of the high concentration diet affected numerous physiological markers for AD (FIG. 5C-D). For example, phosphorylation of tau protein at threonine 231 was significantly reduced, as measured by AT180 levels (FIG. 5D).

Taken together, these results show that the inventive combination diet is effective at preventing cognitive decline associated with AD pathology in 2 different mouse models of AD. Furthermore, the diet has disease-modifying properties. This combination diet thus represents a highly promising treatment for human AD and meets an urgent need in the art for AD therapies, especially ones, such as this one, that are likely to be extremely safe and well tolerated in humans.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

1-31. (canceled)

32. A pharmaceutical composition, comprising at least about 15 mg/kg patient body weight curcumin, at least about 0.1 mg/kg patient body weight piperine, at least about 7.0 mg/kg patient body weight epigallocatechin-3-gallate, at least about 6.0 mg/kg patient body weight N-acetylcysteine, at least about 3.0 mg/kg patient body weight α-lipoic acid.

33. The pharmaceutical composition of claim 32, comprising from about 15 to about 170 mg/kg patient body weight curcumin, from about 7.5 to about 85 mg/kg patient body weight epigallocatechin-3-gallate, from about 6.4 to about 71 mg/kg patient body weight N-acetylcysteine, and from about 3.5 to about 43 mg/kg patient body weight α-lipoic acid.

34. The pharmaceutical composition of claim 32, comprising from about 17 to about 50 mg/kg patient body weight curcumin, at least about 0.5 mg/kg patient body weight piperine, from about 8.5 to about 25 mg/kg patient body weight epigallocatechin-3-gallate, from about 7 to about 21 mg/kg patient body weight N-acetylcysteine, and from about 4 mg/kg to about 13 mg/kg patient body weight α-lipoic acid.

35. The pharmaceutical composition of claim 32, further comprising one or more of:
at least about 0.6 mg/kg patient body weight vitamin B₆;
at least about 0.5 mg/kg patient body weight vitamin B₁;
at least about 1.0 μg/kg patient body weight vitamin B₁₂;
at least about 3.0 μg/kg patient body weight folate; and
at least about 0.3 mg/kg patient body weight vitamin C.

36. The pharmaceutical composition of claim 32, further comprising:
at least about 0.6 mg/kg patient body weight vitamin B₆;
at least about 0.5 mg/kg patient body weight vitamin B₁;
at least about 1.0 μg/kg patient body weight vitamin B₁₂;
at least about 3.0 μg/kg patient body weight folate; and
at least about 0.3 mg/kg patient body weight vitamin C.

37. The pharmaceutical composition of claim 35, wherein the vitamin B₁ is in the form of benfotiamine.

38. The pharmaceutical composition of claim 32, wherein the composition is provided in a unit dosage form comprising one unit dosage.

39. The pharmaceutical composition of claim 32, wherein the composition is provided in a unit dosage form comprising more than one unit dosage.

40. A method for treating Alzheimer's disease in a patient in need thereof, comprising administering daily to the patient a therapeutically effective amount of a pharmaceutical composition comprising at least about 15 mg/kg patient body weight curcumin, at least about 0.1 mg/kg patient body weight piperine, at least about 7.0 mg/kg patient body weight epigallocatechin-3-gallate, at least about 6.0 mg/kg patient body weight N-acetylcysteine, and at least about 3.0 mg/kg patient body weight α-lipoic acid.

41. The method of claim 40, wherein the pharmaceutical composition comprises from about 15 to about 170 mg/kg patient body weight curcumin, from about 7.5 to about 85 mg/kg patient body weight epigallocatechin-3-gallate, from about 6.4 to about 71 mg/kg patient body weight N-acetylcysteine, and from about 3.5 to about 43 mg/kg patient body weight α-lipoic acid.

42. The method of claim 40, wherein the pharmaceutical composition comprises from about 17 to about 50 mg/kg patient body weight curcumin, at least about 0.5 mg/kg patient body weight piperine, from about 8.5 to about 25 mg/kg patient body weight epigallocatechin-3-gallate, from about 7 to about 21 mg/kg patient body weight N-acetylcysteine, and from about 4 mg/kg to about 13 mg/kg patient body weight α-lipoic acid.

43. The method of claim 40, wherein the pharmaceutical composition further comprises one or more of:
at least about 0.6 mg/kg patient body weight vitamin B₆;
at least about 0.5 mg/kg patient body weight vitamin B₁;
at least about 1.0 μg/kg patient body weight vitamin B₁₂;
at least about 3.0 μg/kg patient body weight folate; and
at least about 0.3 mg/kg patient body weight vitamin C.

44. The method of claim 40, wherein the pharmaceutical composition further comprises:
at least about 0.6 mg/kg patient body weight vitamin B₆;
at least about 0.5 mg/kg patient body weight vitamin B₁;
at least about 1.0 μg/kg patient body weight vitamin B₁₂;
at least about 3.0 μg/kg patient body weight folate; and
at least about 0.3 mg/kg patient body weight vitamin C.

45. The method of claim 40, wherein the composition is provided in a unit dosage form and the method comprises administering one unit dosage daily.

46. The method of claim 40, wherein the composition is provided in unit dosage form and the method comprises administering more than one unit dosage daily.

47. The method of claim 40, wherein treating Alzheimer's disease comprises treating one or more adverse cognitive symptoms associated with Alzheimer's disease.

48. The method of claim 47, wherein the one or more adverse cognitive symptoms is selected from the group consisting of memory loss, personality change, agitation, disorientation, loss of coordination, inability to care for one's self, and combinations thereof.

49. The method of claim 40, wherein treating Alzheimer's disease comprises treating one or more adverse physiological symptoms associated with Alzheimer's disease.

50. The method of claim 49, wherein the one or more adverse physiological symptoms is selected from the group consisting of amyloid plaques, tau protein tangles, tau protein phosphorylation, microtubule destabilization, synaptic loss, and combinations thereof.

51. The method of claim 40, wherein treating Alzheimer's disease comprises reducing a level of a low molecular weight oligomeric beta amyloid peptide in the patient.

52. The method of claim 51, wherein the oligomeric beta amyloid peptide is Aβ*56.

53. The method of claim 40, wherein treating Alzheimer's disease comprises reducing levels of tau phosphorylation at threonine 231 in the patient.
